(19) United States
(12) Patent Application Publication
(10) Pub. No.: US 2026/0279587 A1
(43) Pub. Date: Sep. 17, 2026

(54) METHODS AND SYSTEMS FOR MEDICAL CONDITION RISK PREDICTION AND PREDICTION TRIGGERING

(71) Applicant: PRENOSIS, INC., Chicago, IL (US)

(72) Inventors: Akhil Bhargava, Chicago, IL (US); Jonah Weiner Ellman, Chicago, IL (US); Lee Alan Schmalz, Chicago, IL (US); Gregory Luke Watson, Vista, CA (US); Carlos German López-Espina, Evanston, IL (US); Bobby Reddy, Jr., Chicago, IL (US)

(21) Appl. No.: 19/472,052

(22) PCT Filed: Apr. 12, 2024
(86) PCT No.: PCT/US2024/024470

§ 371 Date: Oct. 2, 2025

Related U.S. Application Data
(60) Provisional application No. 63/459,521, filed on Apr. 14, 2023.

(51) Int. Cl. G16H 50/30; G16H 10/60

(52) U.S. Cl. CPC G16H 50/30; G16H 10/60

(57) ABSTRACT

Computer-implemented systems and methods are provided for determining when to trigger an algorithm, or other computer workflow or program, based on input feature availability such that the algorithm generates meaningful outputs. The provided methods involve determining whether input features are ready for generating an algorithm output. For instance, an input feature is ready if it has a measured and acceptable value. If one or more of the input features does not have a measured and acceptable value, such as a measured value being absent or outdated, then the provided methods involve determining whether a value can be imputed to the one or more input features without available values and whether such imputable input features are ready for an imputed value. Once all input features are determined to be ready, imputed values are determined for input features as needed and the algorithm is triggered.

### CROSS-REFERENCE TO RELATED APPLICATION

This application claims priority and the benefit of U.S. Provisional Patent Application No. 63/459,521, filed on Apr. 14, 2023, the contents of which are hereby incorporated by reference in their entirety.

### FIELD

The present application relates generally to machine learning algorithms for medical condition risk prediction. More specifically, the present application provides computer-implemented methods for generating a predicted risk of a patient having or developing a medical condition and methods for triggering such prediction.

### BACKGROUND

A challenge faced by clinicians is to assess a patient's progression towards a medical condition such that preventative or treatment measures can be started at an early stage. Another challenge may be to diagnose a medical condition that is difficult to classify. For example, sepsis is a major health problem and an important cause of mortality in intensive care units (ICUs) worldwide. For instance, septic shock is a significant cause of mortality with a mortality rate reaching up to 50%. In spite of the continuous efforts to put solid criteria for early diagnosis of sepsis, physicians still find it a challenge to accurately identify patients with sepsis in wards and/or emergency departments. Previously, the systemic inflammatory response syndrome (SIRS) diagnostic criteria was used to detect sepsis in different clinical situations. The previous consensus definitions of sepsis (Sepsis-2) required the presence of infection, along with at least two criteria for SIRS, as well as organ failure for diagnosis of sepsis. However, it has been claimed that these criteria were neither sensitive nor specific and had hindered the accurate detection and identification of sepsis.

Sepsis can conceptually be described as patient decompensation due to an infection. The currently used tool for determining patient decompensation, and therefore assessing for sepsis, is the Sequential Organ Failure Assessment (SOFA) score. The use of this score for sepsis was based on the new definition of sepsis (Sepsis-3) as “life-threatening organ dysfunction caused by a deregulated host response to infection.” As part of this definition, sepsis can be diagnosed when a patient exhibits an increase of two or more points in the SOFA score. The SOFA score is based on evaluation of the function of six systems, namely the respiratory, neurological, cardiovascular, hepatic, renal, and coagulation systems. A less time-consuming score, the quick SOFA score (qSOFA), has also been developed. The qSOFA score indicates that sepsis is very likely if the patient has any two of the following: Glasgow Coma Scale<15, systolic blood pressure<100 mm Hg, and respiratory rate>22/min.

Sepsis is an acute condition where symptoms are sudden, and sepsis can often lead to adverse outcomes if not treated promptly. There is high treatment efficacy during the initial stages of infection despite the difficulty in detection. However, as the infection causes an aggressive host response that escalates into sepsis, the efficacy of available treatment options diminishes, which highlights the importance of early detection of a patient's risk for developing sepsis. Because SOFA and qSOFA were designed to screen populations and have not been optimized for individual patients, it still remains a challenge to accurately predict a specific patient's risk of having or developing sepsis. Accurately predicting a specific patient's risk of having or developing other medical conditions (e.g., acute medical conditions) faces similar challenges as sepsis.

Additionally, for certain computer algorithms (e.g., machine learning algorithm), or other computer workflows or programs, to provide an output (e.g., prediction), all input features of the algorithm must have a single, available value at the time of output generation. Additionally, the more accurate the values for the input features, the more accurate the output will be. In some applications of computer algorithms, values for the input features may be obtained from different sources, at different times, at different frequencies, over the course of different time periods, etc. Therefore, in such applications, input feature values may be missing or outdated, or there may be multiple values for a single input feature, when a request is made for the computer algorithm to generate an output. For example, machine learning algorithms in the medical field (e.g., for predicting a diagnosis) rely upon physiological measurements and other testing values (e.g., clinical observations) as input features. An input feature may be missing at the time a prediction request is made, for instance, because a laboratory measurement was ordered and does not yet have a result. Another input feature may have an outdated value because the value was obtained days before the prediction request was made and therefore may not reflect a patient's current condition. Yet another input feature may have multiple values because a value is captured every few seconds (e.g., heart rate). The utility of an algorithm may be substantially diminished if it were only able to output a result when all input features are received, primarily because the time from initiation of the algorithm to when it outputted a result could be very long. Conversely, if the algorithm were capable of outputting a result with a subset of the input features, allowing it to output the result earlier in time, there may be instances in which too many critical features are missing at the time of result, which therefore reduces the accuracy of the output. It is therefore a technological challenge to determine when to trigger an algorithm so that it generates a useful output based on the availability of the algorithm's input features that is both (1) accurate enough for the use case and (2) generated in a timely fashion.

### SUMMARY

The present disclosure provides new and innovative computer-implemented methods and systems for generating a clinically accurate risk assessment within a timely manner for patients at risk of having or developing a medical condition, such as an acute medical condition. Generating the clinically accurate risk assessment includes predicting a risk level (e.g., probability) of a patient having or developing the medical condition through the use of a prediction algorithm, such as a machine learning model. In various embodiments, the medical condition may be an acute condition that is severe and sudden in onset, and therefore requires timely treatment, such as sepsis. The machine learning model generates an output (e.g., a prediction) from a set of input features, values of which are determined from various measurements or clinical observations of the patient, or from an imputation process. The methods may include authentication procedures to help prevent unauthorized individuals from gaining access to protected health information of a patient. In some instances, received input features (e.g., patient measurements or clinical observations) may have different parameters than the input features on which the machine learning model was trained. For example, the received input features may be in different units, or evaluated using a different scale or method, than the input features with which the machine learning model was trained because different organizations may utilize different units, scales, or methods. In such instances, a pre-processing method can be used to determine how the received input features are mapped to the input features with which the machine learning model was trained. Mappings determine any transformation functions on those received input features, such as unit or scale/method conversions. Users of the provided system (e.g., administrators) can configure the mappings for a particular organization and/or can configure normal ranges for each input feature for the organization. When input features are displayed to an end-user, if any of the features are outside the configured normal range, then those features are indicated as such.

The provided methods include a method for determining when, or if, to trigger the machine learning model to generate a prediction. The triggering method is based on input feature availability so that the machine learning model generates meaningful outputs in a timely manner rather than generating an output from incomplete information or waiting a long time to receive one or more input features. In certain instances, the triggering method involves imputing values to input features missing measured values according to predetermined logic designed so that the machine learning model still generates meaningful outputs with the imputed value(s). The triggering method is also designed to strike the right balance between causing the algorithm to output in a timely manner and maintaining appropriate accuracy of the output. The triggering method may be used with any suitable algorithm (e.g., a sepsis prediction machine learning model), or other suitable computer workflow or program.

In various embodiments, the provided methods may include an active ordering method for determining when, or if, to indicate to a clinician (e.g., via a display of a computing device) or hospital electronic medical record that additional parameters should be obtained for a patient in order for the machine learning model to have the highest chance of triggering in a timely and accurate fashion. In certain instances, this active ordering method may involve reading stored data such as the state of information regarding each input feature, including but not limited to: the availability of the feature, the last known measurement of the feature, the last known order time for the feature, and the current time. The active ordering method may include actively alerting a clinician or communicate with a hospital electronic medical record to trigger prompt ordering of necessary labs or patient evaluations to provide necessary data to the machine learning model in as timely a fashion as possible. The active ordering method may involve communicating with the triggering method to indicate that an input feature is expected to be available in a timely fashion, which could cause the triggering method to adjust the time windows for waiting for the missing features before resulting.

In various embodiments, the provided methods may include feature importance methods for indicating to the end user the contribution of each feature to the final prediction result that is displayed to the end user. These feature importance methods allow for the user to understand, on a patient-by-patient basis, which features were most important in creating the prediction result. These feature importance methods may include displaying to the end user which features were missing and imputed, which features were not used, and/or which features were used by the machine learning model to calculate the prediction result. These feature importance methods may further include displaying to the end user the amount that each feature, whether imputed or not, either increased, decreased, or had no effect upon the predicted risk of the patient having the medical condition of interest.

The provided methods may include monitoring for model drift of the machine learning model. Model drift may be monitored indirectly by monitoring for data drift. Various tests are performed to identify the possible occurrence of data drift of the machine learning model. Data drift reports may be generated that show the results of the tests used. If at least one of the tests is failed, the data drift report indicates that data drift is detected. An end-user may then use the data drift report to determine whether data drift has truly occurred, and if so, to determine corrective action to take.

In an example, a computer-implemented method includes receiving a plurality of input features of a machine learning model. The machine learning model is configured to generate the prediction of the risk of the patient having or developing the medical condition. The method further includes determining whether each of the plurality of input features is in a first state or a second state. The first state and the second state are indicative of whether an input feature of the plurality of input features is available for triggering the machine learning model. Each of the plurality of input features has a respective set of requirements to be in the first state. The method further includes generating, using the machine learning model and subsequent to determining that each of the plurality of input features is in the first state, the prediction of the risk of the patient having or developing the medical condition.

It is noted that while the term “clinician” is used throughout this disclosure, it is not intended that the present techniques are limited to clinicians. Rather, any suitable healthcare professional may use, be involved with, etc. the present techniques. For example, a nurse, physician's assistant, medical resident, medical student, etc. could initiate a request for algorithm prediction or for a measurement order at their own discretion or at the instruction of a clinician.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates a box diagram of a system for triggering an algorithm stored on a computing device, according to an aspect of the present disclosure.

FIG. 2 illustrates a flow chart of a method for generating a predicted risk level of a patient for having or developing a medical condition, according to an aspect of the present disclosure.

FIG. 3 illustrates an example user interface that displays the predicted risk level, according to an aspect of the present disclosure.

FIG. 4 illustrates a flow chart of a method for triggering an algorithm stored on a computing device, according to an aspect of the present disclosure.

FIG. 5A illustrates a flow chart of a method for determining a state of an input feature included in the method of FIG. 4, according to an aspect of the present disclosure.

FIG. 5B illustrates a flow chart of an example active ordering method included in the method of FIG. 5A, according to an aspect of the present disclosure.

FIG. 6 illustrates a chart of example state determinations for seven input features, according to an aspect of the present disclosure.

FIG. 7 illustrates a box diagram of a computer system, according to an aspect of the present disclosure.

### DETAILED DESCRIPTION

In general, the present application provides new and innovative systems and computer-implemented methods for predicting, using a prediction algorithm (e.g., machine learning model), a risk level (e.g., probability) of a patient having or developing a medical condition. In various embodiments, the medical condition may be an acute condition that is severe and sudden in onset, and therefore requires timely treatment, such as sepsis. The remaining description references a risk of a patient having or developing sepsis as an example to illustrate the concepts described herein, though it will be appreciated that the concepts described herein may be applicable to other suitable medical conditions, such as the non-exhaustive examples including acute kidney injury, acute respiratory distress syndrome, stroke, asthma attacks, burns, heart attack, infection, and trauma. In at least some embodiments, upon receiving a request (e.g., from a clinician's input device) to trigger the machine learning model to start the process of generating a prediction, the provided systems begin listening for input feature values from a variety of measurement devices or user input devices, such as by reading data stored in a memory of the provided systems. The stored data is continually updated as new information is received from the measurement devices or user input devices. Stated differently, in such embodiments, the provided systems continually receive and store input features, but the systems do not execute the triggering method that involves reading (e.g., by the systems' processor) the stored input features until a request is received.

Once the triggering method is executed, the stored input features are read until either the machine learning model generates a prediction or it is determined that the machine learning model is unable to generate a prediction based on the available input feature values. In other embodiments, the triggering method may involve continually reading the stored input feature. In such embodiments, the triggering method may wait to proceed further in the method than reading the stored input features until the request is received. An input feature collected by the measurement devices or user input devices is received at the provided system (e.g., computing device) near instantaneously as the input feature value is collected (e.g., measured) such that the provided systems receive input features in real-time. As the input features are collected, the triggering method determines if and when all of the input features to the machine learning model have an available value so that a prediction can be generated, which may include imputing a value to an input feature. The trigger logic is described in more detail below.

In various embodiments, the computer-implemented methods include authentication and authorization procedures. The authentication and authorization procedures are managed through the configuration of users, access policies, and authentication clients, and are designed to help prevent unauthorized individuals from gaining access to a patient's protected health information under the Health Insurance Portability and Accountability Act of 1996 (HIPAA). The authentication and authorization procedures may be implemented prior to enabling a user to initiate a request to trigger the machine learning model or may be implemented after receiving the request and prior to proceeding with the prediction generation.

In various embodiments, at least some of the received input feature values may be pre-processed, prior to inputting the input features into the machine learning model, to match a set of parameters of the input features on which the machine learning model was trained. For instance, received input feature values may have different parameters than the input features on which the machine learning model was trained. For example, the received input feature values may be in different units, or evaluated using a different scale or method, than the input features on which the machine learning model was trained because different organizations (e.g., hospitals or other healthcare facilities) may utilize different units, scales, or methods. In such instances, a pre-processing technique can be used to determine how the received input feature values are mapped to the input features with which the machine learning model was trained. Mappings determine any transformation functions on those received input feature values, such as unit or scale/method conversions. For example, in various embodiments, received input feature values may be mapped by Logical Observation Identifiers Names and Codes (LOINC) code to the appropriate model input feature. A LOINC code, as is known to a person having skill in the art, is a universal standard for identifying medical laboratory observations that facilitates the exchange and pooling of results such as laboratory tests or vital signs for clinical care, outcomes management, and research. Users of the provided system (e.g., administrators) can configure the mappings for a particular organization and/or can configure normal ranges for each input feature for the organization.

As stated above, the prediction method includes a new and innovative triggering method (e.g., trigger logic executable by a processor) for determining when to trigger the prediction algorithm (e.g., machine learning model) based on input feature availability such that the prediction machine learning model generates meaningful (e.g., accurate) outputs, which may include that the output of the prediction machine learning model is “no result” so that a clinician is notified in a timely manner that the clinician cannot rely upon a prediction at that time based on the available input features and must resort to manual diagnosis or to requesting a prediction a second time. While the following description of the triggering method is in relation to algorithms or machine learning models, the concepts described herein can also be applied to suitable computer workflows or programs that require timely input features to generate an output. The provided trigger logic involves determining whether input features are in a first state indicative of an input feature being ready (e.g., has an available value) for generating an algorithm output. For instance, an input feature is in the first state if it has a measured and acceptable value. If one or more of the input features does not have a measured and acceptable value, such as a measured value being absent or outdated, then the provided trigger logic involves determining whether a value can be imputed to the one or more input features without available values and whether such imputable input features are in the first state such that they be may be imputed with a substitute value. Stated differently, if an imputable input feature is in the first state, then the imputable input feature has an available value (i.e. the value that will be imputed).

Imputation is the process of replacing missing data with substituted values. Imputation has the benefit of providing values for all input features necessary for an algorithm to provide a result. However, there is no guarantee a substituted value is an accurate representation of the true value (e.g., measured value) of the missing input feature and therefore a true value is preferred even if it might take longer to obtain the true value as compared to the substitute value. The provided trigger logic defines an imputation strategy that balances the need for important input features, the need for less important input features, and the time to receive values for input features (e.g., features ordered by physicians). For instance, certain input features may be deemed too important to the accuracy of the algorithm prediction and therefore values may not be imputed to these important input features. As such, if these important input features do not have measured values, then the algorithm will not be triggered to generate a prediction. For other input features that can be imputed, the provided trigger logic may include waiting a predetermined amount of time to receive a measured value before imputing a value. The imputation strategy of the provided trigger logic will be described in more detail below.

In various aspects, if a value cannot be imputed to one or more input features missing available values, then the provided trigger logic may refrain from triggering the algorithm. Otherwise, in such aspects, an imputed value is determined for each of the missing input features and the algorithm is thereafter triggered to generate an output from the available values including input features and imputed input features. Refraining from triggering the algorithm until all input features have available values in these aspects, and only imputing certain input features in certain instances, results in consistently meaningful (e.g., accurate) outputs from the algorithm. In other aspects, the algorithm may be triggered when one or more input features that cannot be imputed are missing. For instance, it may be predetermined, or determined as part the triggering method, that a certain combination of input features, or of input features and imputed input features, will generate a consistently meaningful (e.g., accurate) prediction without the one or more missing input features.

While the provided trigger logic can be implemented with any suitable computer algorithm having input features, one example implementation is with a prediction algorithm in the medical context, such as the machine learning algorithm trained to predict a risk of a patient having or developing sepsis. Measurement of patient-specific input features for an algorithm depends upon the clinical setting (e.g., hospital ordering policies) and healthcare provider (e.g., physician) decisions, resulting in substantial variation in the availability, frequency, and timeliness of the patient-specific input features. For instance, a patient might never have a measured value for an input feature because a physician chooses to never order a comprehensive metabolic panel for the patient. In another instance, a patient may have multiple measured values for an input feature because a heart rate monitor provides high-frequency measurements. In another instance, a patient may have a measured value for an input feature that was measured much earlier (e.g., four days) than a request is received for an algorithm prediction, and therefore the measured value may not provide an accurate representation of the patient's current health. In another instance still, a measured value for an input parameter might not be available until much later (e.g., two days) than when a request is received for an algorithm prediction because it takes time for laboratory tests to be completed. All of these example variations in input feature availability pose challenges in promptly generating a meaningful (e.g., clinically accurate) algorithm prediction of whether the patient has sepsis or is at risk of developing sepsis. As will be described in detail below, the provided methods address these challenges.

The remaining description of the provided trigger logic, or trigger method, will be described with reference to the example algorithm or machine learning model for predicting a patient's risk of having or developing sepsis to help illustrate the aspects of the trigger logic, or trigger method, described herein, though as stated above, the provided trigger logic, or trigger method, is applicable to any suitable algorithm, computer workflow, or program.

In various embodiments, the provided methods may include active ordering logic that may be executed by a processor (e.g., the processor 122) for determining when, or if, to indicate to a clinician (e.g., via a display of a computing device) or hospital electronic medical record that additional parameters should be obtained for a patient in order for the machine learning model to have the highest chance of triggering in a timely and accurate fashion. In certain instances, this active ordering logic may read stored data such as the state of information regarding each input feature, including but not limited to: the availability of the feature, the last known measurement of the feature, the last known order time for the feature, and the current time. This active ordering logic may actively alert a clinician or communicate with a hospital electronic medical record to trigger prompt ordering of necessary labs or patient evaluations to provide necessary data to the machine learning model in as timely a fashion as possible. This active ordering logic may communicate with the triggering method to indicate that an input feature is expected to be available in a timely fashion, which could cause the triggering method to adjust the time windows for waiting for the missing features before resulting.

In various embodiments, the provided methods may include feature importance methods for indicating to the end user the contribution of each feature to the final prediction result that is displayed to the end user. These feature importance methods allow for the user to understand, on a patient-by-patient basis, which features were most important in creating the prediction result. These feature importance methods may include displaying to the end user which features were missing and imputed, which features were not used, and/or which features were used by the machine learning model to calculate the prediction result. These feature importance methods may further include displaying to the end user the amount that each feature, whether imputed or not, either increased, decreased, or had no effect upon the predicted risk of the patient having the medical condition of interest.

In various embodiments, the provided methods include determining whether the machine learning model is demonstrating model drift. Model drift is the degradation of a model's predictive performance due to systemic or statistical changes, which may be the result of data drift and/or distribution shift, though not every detected data drift or distribution shift will cause a degradation in the machine learning model's predictive performance. Data drift relates to unexpected or undocumented changes to data structures, semantics, and infrastructure that is a result of modern data architectures. These changes are irrespective of the training dataset and are often caused by issues within the data collection process. For instance, examples of data drift include: (1) a site at which the machine learning model is implemented making an undocumented change from one LOINC code to a new LOINC code for an input parameter, (2) the machine learning model began not receiving heart rate measurements from the site because of a failed deployment of an IT system at the site, (3) uncalibrated temperature probes are used that cause a shift in a temperature reading, and (4) values stored for use with the machine learning model are incorrectly truncated. Detecting data drift may include generating a data drift report, which is a report to detect potential data drift showing the result of statistical tests comparing a reference dataset to an observed dataset. In various embodiments, model drift may be monitored indirectly by monitoring for data drift using methods provided herein.

Distribution shift relates to an unexpected change in the input, label, and/or relationship of input and label features in newly observed data that does not match the training dataset of the machine learning model. A distribution shift occurs when the training data of the machine learning model is no longer representative of newly observed data. The events causing a distribution shift are due to environmental or population changes and not software issues. For instance, examples of distribution drift include: (1) a new machine being able to obtain a measurement for an input feature at a level lower than what is observed in the training dataset, (2) the incidence of septic patient changes in a new observed dataset, and (3) a new drug reducing procalcitonin (PCT) levels that becomes routinely measured for septic patients, thus changing the relationship identified in the training dataset.

### Prediction System

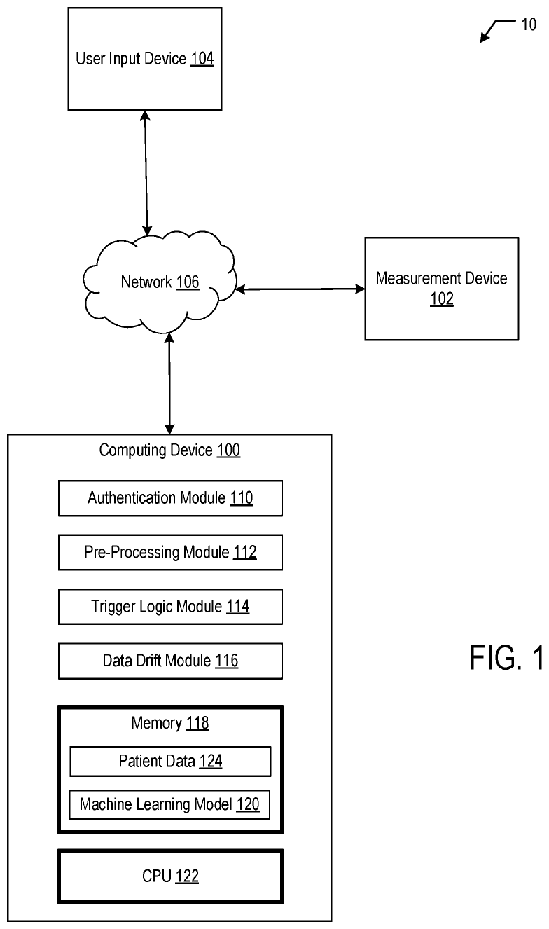

FIG. 1 illustrates an example computer network 10 that may be used to implement various aspects of the present application. Generally, the computer network 10 includes various devices communicating and functioning together in the gathering, transmitting, requesting and/or processing of data related to generating an output of an algorithm stored on a computing device. As illustrated, a communications network 106 allows for communication in the computer network 10. The communications network 106 may include one or more wireless networks such as, but not limited to one or more of a Local Area Network (LAN), Wireless Local Area Network (WLAN), a Personal Area Network (PAN), Campus Area Network (CAN), a Metropolitan Area Network (MAN), a Wide Area Network (WAN), a Wireless Wide Area Network (WWAN), Global System for Mobile Communications (GSM), Personal Communications Service (PCS), Digital Advanced Mobile Phone Service (D-Amps), Bluetooth, Wi-Fi, Fixed Wireless Data, 2G, 2.5G, 3G, 4G, LTE networks, enhanced data rates for GSM evolution (EDGE), General packet radio service (GPRS), enhanced GPRS, messaging protocols such as, TCP/IP, SMS, MMS, extensible messaging and presence protocol (XMPP), real time messaging protocol (RTMP), instant messaging and presence protocol (IMPP), instant messaging, USSD, IRC, or any other wireless data networks or messaging protocols. The communications network 106 may also include wired networks.

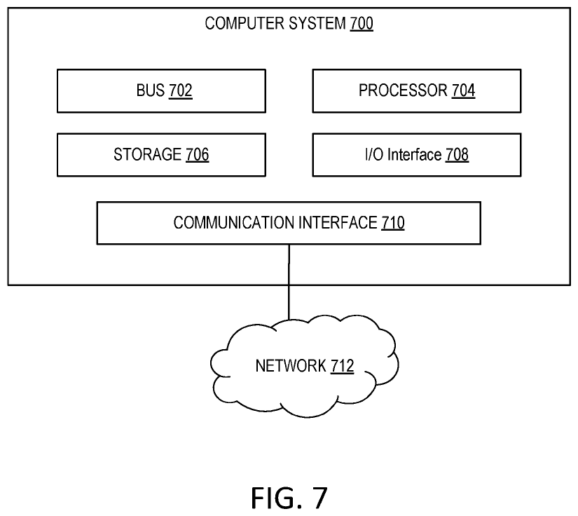

The example computer network 10 includes a computing device 100 that is in communication with one or more measurement devices 102 and one or more user input devices 104. The computing device 100 may be a server or other suitable computer system having a memory 118 and a processor 122 such as the computer system 700 (FIG. 7). The memory 118 stores an algorithm (e.g., machine learning model 120). Alternatively, the machine learning model 120 may be stored on a server or other suitable computing device in communication with the computing device 100. The machine learning model 120 may be a suitable supervised, semi-supervised, or unsupervised machine learning model. For example, the machine learning model 120 may be one of the machine learning models in the group including random forest, logistic regression, gradient boosting machine, neural networks, support vector machines, k-means clustering, and hierarchical clustering models. In one example, a random forest model was trained by maximizing the Area Under the Receiver Operating Characteristic curve (AUROC) between the predictions and training label. In at least some embodiments, the memory 118 further stores patient data 124. The patient data 124 may include any data of the patient that is related to the input features of the machine learning model 120. The patient data 124 may be continually updated as new data is received from various measurement devices 102 and user input devices 104.

In various embodiments, the computing device 100 includes an authentication module 110, a pre-processing module 112, a trigger logic module 114, and a data drift module 116. Each of the authentication module 110, pre-processing module 112, trigger logic module 114, and data drift module 116 may be implemented by software executed by the processor 122.

A measurement device 102 may be any suitable device that collects and transmits data. In an example, a measurement device 102 may be a heart rate monitor that collects information on a patient's heart rate and transmits that information to the computing device 100. In other examples, a measurement device 102 may be any suitable machine that collects, or is in communication with a machine that collects, information (e.g., values, such as measurements or clinical observations, for any of the input features for the machine learning model 120 described below) relevant to determining a risk of a patient having or developing sepsis. As it is used hereafter, the term “measurement” and its plural form encompasses manual measurements, machine-derived measurements, and clinical observations.

A user input device 104 may be any suitable device, such as a desktop computer, capable of receiving information via user input and transmitting that information. For example, a physician may order a comprehensive metabolic panel for a patient from a first user input device 104 (e.g., a tablet computer) and the laboratory technician may enter the results of the panel into a second user input device 104 (e.g., a desktop computer). In this example, an indication that the order was placed is transmitted from the first user input device 104 to the computing device 100 and the panel results are transmitted from the second user input device 104 to the computing device 100.

The machine learning model 120 generates an output from a plurality of input features. For example, the machine learning model 120 may generate a prediction for a risk level of a patient having or developing a medical condition, such as sepsis, from a plurality of input features. Values of the plurality of input features may be received at the computing device 100 from the one or more measurement devices 102 and/or the one or more user input devices 104 and stored in the memory 118 as patient data 124. For instance, the input features are measured or input information of a patient.

The input features of the machine learning model 120 include at least some (e.g., all) of demographic measurements, patient assessments, vital signs, hematology laboratory values, chemistry laboratory values, and sepsis-specific biomarker concentrations. In various embodiments, the input features include or consist of some or all of the input features in the group consisting of: age, sex at birth, race, ethnicity, past medical history of the patient, current complaints or symptoms of the patient, neurological assessments (e.g., Glasgow Coma Scale, Full Outline of Unresponsiveness (FOUR), etc.), clinical decision support alerts, clinician or other chart notes, diagnosis codes (e.g., ICD-10), procedures performed on the patient (including CPT codes), current medications of the patient (including NDC codes), interventions, patient care setting (e.g., ICU, ED, hospital floor, outpatient clinic, etc.), imaging (e.g., X-ray, CT scan, MRI, fMRI, or ultrasound), electrograms (e.g., EEG or EKG), endoscopic tests, systolic blood pressure, diastolic blood pressure, body temperature, respiratory rate, heart rate, blood oxygen saturation, a fraction of inspired oxygen (FiO2), white blood cell count, lymphocyte count, neutrophil count, platelet count, creatinine, blood urea nitrogen, potassium, chloride, total carbon dioxide, sodium, albumin, bilirubin, procalcitonin, C-reactive protein, lactate, glucose, calcium, hematocrit, hemoglobin concentration, lab-derived biomarkers (e.g., monocyte distribution width, Cytovale® IntelliSep®), clinical severity measures and composites (e.g., Sequential Organ Failure Assessment (SOFA) score, SOFA component scores, Charlson Comorbidity Index, or Acute Physiology and Chronic Health Evaluation (APACHE) I and II), biopsies results, microorganism tests (e.g., cultures, PCR tests, or antigen tests), protein biomarkers (e.g., Angiopoietin-1, Angiopoietin-2, C-Reactive Protein, Cystatin C, D-Dimer, E-Selectin, Fractalkine, FLT3-Ligand, GCSF, GDF15, GMCSF, Granzyme-B, IFN-alpha, IFN-gamma, IL1-beta, IL1-RA, IL-2, IL-4, IL-6, IL-7, IL-8, IL-10, IL-15, IP-10, lactate dehydrogenase (LDH), lipopolysaccharide-binding protein (LBP), Leptin, MCP1, MIP1-alpha, MIP1-beta, MIP3-alpha, NGAL, Pancreatic Stone Protein, PDL-1, Pentraxin-3, Procalcitonin, Protein C, S100b, TGF-alpha, Thromodulin, Tissue Factor, TNF-alpha, TRAIL, TREM-1, Troponin, VCAM-1, and/or VEG-F), genomic biomarkers (e.g., SNPs in CD14, TLR1, TLR2, TLR4, TLR6, TNF-alpha, IL-6, factor V, factor XII, FER, MAN2A1), gene expression or transcriptomic biomarkers (e.g., CEACAM4, LAMP1, PLAC8, PLA2G7, CEACAM1, ZDHHC19, C9orf95, GNA15, BATF, C3AR1, KIAA1370, TGFBI, MTCH1, RPGRIP1, HLA-DPB1, Nuclear factor-kappa B (NF-KB), Heme oxygenase-1 (HO-1), FCGR2C, CRP, IL-6, IL-10, IL-12, TNF, LY6E, LAMP3, ISG15, USP18, RSAD2, IFI44L, NCOA7, CMPK2, IFI44, BATF2, GBP4, IFIH1, GMPR, CXCL10, IFI27, ISG15, CCL2, OAS3, P2RY14, GCH1, CD274, ID01, ARL4A, BPGM, TNFAIP3, TNFAIP6, IL1R1, PROK2, RGS1, CXCR6, RTP4, TLR3, FZD5, KCNJ2, IRAK2, RGS16, CXCL8, OLR1, CCRL2, SPP1, ST3GAL4, CST7, IL1R2, IL4R, GALM, CTLA4, SOCS1, BCL2L1, TGM2, SLC39A8, CA2, PNP, GATA1, SLC1A5, RGS16, CD44, PDGFC, CSF2RA, CXCL1, CSF3R, SOCS3, IL18R1, PIM1, LEPR, IL1B, CSF2RB, ITGB3, STAT1, FAS, EFNA1, IER3, EETS2, SLC2A3, BCL2A1, PFKFB3, CEBPB, BIRC2, ATF3, TUBB2A, G0S2, MERTK, BIK, QSOX1, PYGL, TGFA, P4HA2, LDHA, IRS2, TSPO, HGF, GADD45A, TGFBR3, CD38, PRF1, FASLG, TIMP3, ANKH, LGALS3, PPP2R5B, GPX1, BNIP3L, BCL2L1, KLF7, FOSL2, ADM, MXI1, SELENBP1, PGF, FOXO3, NEDD4L, SLC2A1, SIAH2, MMP9, CXCR3, CD8A, IFNG, CD8B, JAK2, CCL4, ICAM1, WARS1, KRT1, GPR65, DYRK3, ACHE, ARG1, LCN2, LTF, OLFM4, HLA-DMB, YKT6, PDE4B, TWISTNB, BTN2A2, ZBTB33, PSMB9, CAMK4, TMEM19, SLC12A7, TP53BP1, PLEKHO1, SLC25A22, FRS2, CD24, STX1A, KCNMB4, CRISP2, HTRA1, PPL, RHBDF2, ZCCHC4, DDX6, SENP5, RAPGEF1, DTX2, RELB, DYRK2, CCNB1IP1, TDRD9, ZAP70, ARL14EP, MDC1, and/or ADGRE3), or composite biomarker scores (e.g., SeptiCyte® LAB and SeptiCyte® RAPID, Inflammatix® Tri Verity®, or MeMed BV®).

In various aspects, one or more of the input features in the above group may encompass different variations of the input feature (e.g., an input feature derived from another input feature). For example, the heart rate input feature may have variations including a maximum heart rate in the last two hours, a change in heart rate from 6 hours ago, a variance of heart rate measurements over the last hour, one or more features extracted from a waveform analysis of heart rate monitor readings, or other suitable variations. In some aspects, the input features of the machine learning model 120 may include more than one variation of an input feature. For example, the input features of the machine learning model 120 may include both the maximum heart rate in the last two hours and the variance of heart rate measurements over the last hour.

In one example, the input features of the machine learning model 120 include at least one input feature of a first group, at least on input feature of a second group, and at least one input feature of a third group.

The first group includes the following input features: age, sex at birth, race, ethnicity, past medical history of the patient, current complaints or symptoms of the patient, neurological assessments (e.g., Glasgow Coma Scale, Full Outline of Unresponsiveness (FOUR), etc.), clinical decision support alerts, clinician or other chart notes, diagnosis codes (e.g., ICD-10), procedures performed on the patient (including CPT codes), current medications of the patient (including NDC codes), interventions, patient care setting (e.g., ICU, ED, hospital floor, outpatient clinic, etc.), imaging (e.g., X-ray, CT scan, MRI, fMRI, or ultrasound), electrograms (e.g., EEG or EKG), endoscopic tests, systolic blood pressure, diastolic blood pressure, body temperature, respiratory rate, heart rate, blood oxygen saturation, and a fraction of inspired oxygen (FiO2).

The second group includes the following input features: white blood cell count, lymphocyte count, neutrophil count, platelet count, creatinine, blood urea nitrogen, potassium, chloride, total carbon dioxide, sodium, albumin, bilirubin, lactate, glucose, calcium, hematocrit, hemoglobin concentration, lab-derived biomarkers (e.g., monocyte distribution width, Cytovale® IntelliSep®), clinical severity measures and composites (e.g., Sequential Organ Failure Assessment (SOFA) score, SOFA component scores, Charlson Comorbidity Index, or Acute Physiology and Chronic Health Evaluation (APACHE) I and II), biopsies results, and microorganism tests (e.g., cultures, PCR tests, or antigen tests).

The third group includes the following input features: procalcitonin, C-reactive protein, protein biomarkers (e.g., Angiopoietin-1, Angiopoietin-2, C-Reactive Protein, Cystatin C, D-Dimer, E-Selectin, Fractalkine, FLT3-Ligand, GCSF, GDF15, GMCSF, Granzyme-B, IFN-alpha, IFN-gamma, IL1-beta, IL1-RA, IL-2, IL-4, IL-6, IL-7, IL-8, IL-10, IL-15, IP-10, lactate dehydrogenase (LDH), lipopolysaccharide-binding protein (LBP), Leptin, MCP1, MIP1-alpha, MIP1-beta, MIP3-alpha, NGAL, Pancreatic Stone Protein, PDL-1, Pentraxin-3, Procalcitonin, Protein C, S100b, TGF-alpha, Thromodulin, Tissue Factor, TNF-alpha, TRAIL, TREM-1, Troponin, VCAM-1, and/or VEG-F), genomic biomarkers (e.g., SNPs in CD14, TLR1, TLR2, TLR4, TLR6, TNF-alpha, IL-6, factor V, factor XII, FER, MAN2A1), gene expression or transcriptomic biomarkers (e.g., CEACAM4, LAMP1, PLAC8, PLA2G7, CEACAM1, ZDHHC19, C9orf95, GNA15, BATF, C3AR1, KIAA1370, TGFBI, MTCH1, RPGRIP1, HLA-DPB1, Nuclear factor-kappa B (NF-KB), Heme oxygenase-1 (HO-1), FCGR2C, CRP, IL-6, IL-10, IL-12, TNF, LY6E, LAMP3, ISG15, USP18, RSAD2, IFI44L, NCOA7, CMPK2, IFI44, BATF2, GBP4, IFIH1, GMPR, CXCL10, IFI27, ISG15, CCL2, OAS3, P2RY14, GCH1, CD274, ID01, ARL4A, BPGM, TNFAIP3, TNFAIP6, IL1R1, PROK2, RGS1, CXCR6, RTP4, TLR3, FZD5, KCNJ2, IRAK2, RGS16, CXCL8, OLR1, CCRL2, SPP1, ST3GAL4, CST7, IL1R2, IL4R, GALM, CTLA4, SOCS1, BCL2L1, TGM2, SLC39A8, CA2, PNP, GATA1, SLC1A5, RGS16, CD44, PDGFC, CSF2RA, CXCL1, CSF3R, SOCS3, IL18R1, PIM1, LEPR, IL1B, CSF2RB, ITGB3,STAT1, FAS, EFNA1, IER3, EETS2, SLC2A3, BCL2A1, PFKFB3, CEBPB, BIRC2, ATF3, TUBB2A, G0S2, MERTK, BIK, QSOX1, PYGL, TGFA, P4HA2, LDHA, IRS2, TSPO, HGF, GADD45A, TGFBR3, CD38, PRF1, FASLG, TIMP3, ANKH, LGALS3, PPP2R5B, GPX1, BNIP3L, BCL2L1, KLF7, FOSL2, ADM, MXI1, SELENBP1, PGF, FOXO3, NEDD4L, SLC2A1, SIAH2, MMP9, CXCR3, CD8A, IFNG, CD8B, JAK2, CCL4, ICAM1, WARS1, KRT1, GPR65, DYRK3, ACHE, ARG1, LCN2, LTF, OLFM4, HLA-DMB, YKT6, PDE4B, TWISTNB, BTN2A2, ZBTB33, PSMB9, CAMK4, TMEM19, SLC12A7, TP53BP1, PLEKHO1, SLC25A22, FRS2, CD24, STX1A, KCNMB4, CRISP2, HTRA1, PPL, RHBDF2, ZCCHC4, DDX6, SENP5, RAPGEF1, DTX2, RELB, DYRK2, CCNB1IP1, TDRD9, ZAP70, ARL14EP, MDC1, and/or ADGRE3), or composite biomarker scores (e.g., SeptiCyte® LAB and SeptiCyte® RAPID, Inflammatix® Tri Verity®, or MeMed BV®).

In an example, the input features of the machine learning model 120 include age, diastolic blood pressure, heart rate, pulse oximetry, respiratory rate, systolic blood pressure, and temperature from the first group; white blood cell count, lymphocyte count, neutrophil count, platelet count, creatinine, blood urea nitrogen, potassium, chloride, total carbon dioxide, sodium, albumin, bilirubin, and lactate from the second group; and procalcitonin and C-reactive protein from the third group.

In another example, the input features of the machine learning model 120 include age, diastolic blood pressure, heart rate, pulse oximetry, respiratory rate, systolic blood pressure, and temperature from the first group; white blood cell count, lymphocyte count, neutrophil count, platelet count, creatinine, blood urea nitrogen, potassium, chloride, total carbon dioxide, sodium, albumin, bilirubin, and lactate from the second group; and procalcitonin, C-reactive protein, and IL-6 from the third group.

In another example, the input features of the machine learning model 120 include age, diastolic blood pressure, heart rate, pulse oximetry, respiratory rate, systolic blood pressure, and temperature from the first group; white blood cell count, lymphocyte count, neutrophil count, platelet count, creatinine, blood urea nitrogen, potassium, chloride, total carbon dioxide, sodium, albumin, bilirubin, and lactate from the second group; and procalcitonin, C-reactive protein, angiopoietin-2, IL-6, IL-8, IP-10, and pentraxin-3 from the third group.

In another example, the input features of the machine learning model 120 include age, diastolic blood pressure, heart rate, pulse oximetry, respiratory rate, systolic blood pressure, and temperature from the first group; white blood cell count, lymphocyte count, neutrophil count, platelet count, creatinine, blood urea nitrogen, potassium, chloride, total carbon dioxide, sodium, albumin, bilirubin, and lactate from the second group; and procalcitonin, C-reactive protein, angiopoietin-2, IL-6, IL-8, IP-10, PDL-1, TREM-1, and pentraxin-3 from the third group.

In another example, the input features of the machine learning model 120 include age, diastolic blood pressure, heart rate, pulse oximetry, respiratory rate, systolic blood pressure, and temperature from the first group; white blood cell count, lymphocyte count, neutrophil count, platelet count, creatinine, blood urea nitrogen, potassium, chloride, total carbon dioxide, sodium, albumin, bilirubin, and lactate from the second group; and procalcitonin, C-reactive protein, angiopoietin-1, angiopoietin-2, E-Selectin, Fractalkine, FLT3-Ligand, GCSF, GMCSF, Granzyme-B, IFN-alpha, IFN-gamma, IL1-beta, IL1-RA, IL-2, IL-4, IL-6, IL-7, IL-8, IL-10, IL-15, IP-10, Leptin, MCP1, MIP1-alpha, MIP1-beta, MIP3-alpha, NGAL, PDL-1, Pentraxin-3, TGF-alpha, Thromodulin, Tissue Factor, TNF-alpha, TRAIL, TREM-1, VCAM-1, and VEG-F from the third group.

In another example, the input features of the machine learning model 120 include age, diastolic blood pressure, heart rate, pulse oximetry, respiratory rate, systolic blood pressure, sex at birth, and temperature from the first group; creatinine, blood urea nitrogen, potassium, chloride, total carbon dioxide, sodium, calcium, hematocrit, and hemoglobin concentration from the second group; and angiopoietin-2, IL-8, IP-10, and pentraxin-3 from the third group.

Demographic measurements include age, sex at birth, race, and ethnicity. Age can be calculated as the current date minus the patient's date of birth. Age has been traditionally considered a risk factor, as the incidence of sepsis increases as patients age. This may be associated with an increase in chronic conditions that are important in the progression or presence of sepsis.

Patient assessments include past medical history of the patient, current complaints or symptoms of the patient, neurological assessments (e.g., Glasgow Coma Scale, Full Outline of Unresponsiveness (FOUR), etc.), clinical decision support alerts, clinician chart notes or other chart notes, diagnosis codes (e.g., ICD-10) associated with the patient, procedures performed on the patient (including CPT codes), current medications of the patient (including NDC codes), interventions, patient care setting (e.g., ICU, ED, hospital floor, outpatient clinic, etc.), imaging (e.g., X-ray, CT scan, MRI, fMRI, or ultrasound), electrogram data (e.g., EEG or EKG), endoscopic test data, and clinical severity measures and composites (e.g., Sequential Organ Failure Assessment (SOFA) score, SOFA component scores, Charlson Comorbidity Index, or Acute Physiology and Chronic Health Evaluation (APACHE) I and II).

As used herein, clinical decision support alerts are notifications or actions that notify the status of a patient's medical condition. The notification may stem from a result of a software application or a human-driven action. An example of an alert from a software application would be a machine learning algorithm which uses data from a patient's medical record to generate an alert for a patient at high risk of sepsis (e.g., a sepsis early warning system). An example of an alert from a human-driven action would be a health care professional contacting another healthcare professional's communication device regarding a patient's medical condition.

As used herein, interventions are treatments, procedures, or other actions that a healthcare professional may perform to prevent or treat a medical condition or improve a patient's health. Interventions include, but are not limited to, medications (e.g., antibiotics, vasopressors, immunosuppresants, topical creams), surgeries (e.g., amputation), vaccinations, education and behavior training, and environmental alterations.

Glasgow coma scale score is a measurement resulting in a score on a clinical scale used to reliably measure a person's level of consciousness. Altered mental state (e.g., measured by Glasgow Coma Scale (GCS)) is a beneficial category for prediction of sepsis measured in both sequential organ failure assessment (SOFA) score as well as quick SOFA score (qSOFA). For instance, each of the SOFA score and the qSOFA score utilizes the Glasgow coma scale score.

Vital signs include systolic blood pressure, diastolic blood pressure, temperature, respiratory rate, heart rate, and blood oxygen saturation. Systolic blood pressure is the pressure exerted when the heart beats and blood is ejected into the arteries. It can be measured using a machine, such as a sphygmomanometer. Systolic blood pressure is used in the calculation of Mean arterial pressure. Mean arterial pressure is intended to assess the cardiovascular system component of the SOFA score.

Diastolic blood pressure is the pressure within the arteries when the heart rests between beats. It can be measured using a machine, such as a sphygmomanometer. Diastolic blood pressure is used to calculate mean arterial pressure. Mean arterial pressure is intended to assess the cardiovascular system component of the SOFA score.

Temperature is the specific degree of hotness in the body. Temperature can be measured in various ways; one common method is a thermometer. Temperature can be used to identify if a patient has a fever. Fever patterns can be used to identify possible causes of infections, as remittent fevers (fevers that fluctuate more than 1.1° C. and never return to normal) are often caused by infections. Temperature is also used in the systemic inflammatory response syndrome (SIRS) criteria, which is part of the older Sepsis-2 definition.

Respiratory rate is defined as the number of heart beats per minute. Respiratory rate can be measured manually, or through a machine such as a pulse oximeter. An abnormal respiratory rate can be an indication a patient contains an irregular amount of oxygen available. Tachypnea (elevated respiratory rate) is commonly observed within septic patients and is associated with an increase in 28-day mortality in septic patients. Respiratory rate is also associated with SIRS, which is part of the older Sepsis-2 definition.

Heart rate is measured as the number of heartbeats within a certain period. Heart rate can be measured manually, or through a machine such as a heart rate monitor. An irregular heart rate may be associated with poor blood circulation and either hypotension or hypertension. Heart Rate is also associated with SIRS, which is part of the older Sepsis-2 definition.

Blood oxygen saturation is the measure of the amount of oxygen-carrying hemoglobin in the blood relative to non-oxygen-carrying hemoglobin. Blood oxygen saturation can be calculated and measured in many methods, such as by using a pulse oximeter. Blood oxygen saturation is a non-invasive method to estimate the arterial oxygen saturation (SaO₂), which is used in the calculation of PaO₂/FiO₂ of the respiratory system SOFA score calculation.

Hematology laboratory values include white blood cell count, platelet cell count, lymphocyte count, and neutrophil count. White blood cell count is a measure of the number of white blood cells in a patient's blood. White blood cell count can be measured using a hematology laboratory test. An irregularity in white blood cell count can indicate a person is fighting off an infection. White blood cells counts are associated with SIRS, which is a part of the older Sepsis-2 Definition.

Platelet Cell count is a measure of the number of platelet cells in a patient's blood. Platelet cell counts can be measured using a hematology lab test. An irregularity in platelet cell counts can indicate if an individual has difficulty with clotting. Platelets are involved in both hemostasis and the immune response and play a critical role in sepsis. Platelets are used to assess the coagulation system component of the SOFA Score.

Lymphocyte count is a measure of the number of lymphocyte cells in a patient's blood. Lymphocyte cell counts can be measured using a hematology lab test. Lymphocyte cells are a type of white blood cell that is a part of the immune system. An irregularity in the lymphocyte count can indicate the presence of an infection. Lymphocytes combined with neutrophils can be used to calculate the Neutrophil to Lymphocyte Ratio (NLR). NLR is a metric that is associated with mortality in septic patients, as non-survivors exhibit a significantly higher NLR.

Neutrophil count is a measure of the number of neutrophil cells in a patient's blood. Neutrophil cell counts can be measured using a hematology lab test. Neutrophils are a type of granulocyte, which is a type of white blood cell that is a part of the immune system. An irregularity in the lymphocyte count can indicate the presence of an infection. Lymphocytes combined with neutrophils can be used to calculate the Neutrophil to Lymphocyte Ratio (NLR). NLR is a metric that is associated with mortality in septic patients, as non-survivors exhibit a significantly higher NLR.

Hematocrit is the percentage by volume of red blood cells in the blood. Hematocrit percentage can be measured using a hematology lab test. Patients with low hematocrit are associated with higher disease severity. Hematocrit is associated with prognosis for septic patients during admission to ICU.

Hemoglobin concentration is a measure of the hemoglobin protein present in in red blood cells. Hemoglobin concentration can be measured using a hematology lab test. Hemoglobin confers the ability to deliver oxygen to cells. Acute reduction in hemoglobin is associated in patients with sepsis and septic shock.

Chemistry laboratory values include creatinine, blood urea nitrogen, potassium, chloride, total carbon dioxide, sodium, albumin, bilirubin, glucose, calcium, lactate, lab-derived biomarkers (e.g., monocyte distribution width, Cytovale® IntelliSep®), biopsy results, microorganism tests (e.g., cultures, PCR tests, or antigen tests).

Creatinine is defined as the concentration of creatinine within the blood. Creatinine measurements in the blood can be performed using a chemistry laboratory panel. High creatinine in the blood indicates the renal system may not be filtering creatinine into the urine for elimination. Creatinine is used to assess the renal system component of the SOFA Score.

Blood Urea Nitrogen (BUN) is the amount of urea nitrogen that remains in the blood. BUN measurements in the blood can be performed using a chemistry laboratory panel. Urea Nitrogen is a waste compound that is transferred from the blood into the urine by the kidneys. A high BUN may indicate the renal system may not be operating effectively. An elevated BUN has been linked to the development or presence of sepsis.

Potassium is measured as the concentration of potassium ions within the blood. Potassium measurements in the blood can be obtained by performing a chemistry laboratory test. Potassium plays a role in maintaining fluid levels within the body's cells. An imbalance in potassium can result in an imbalance in fluid levels. Disturbances in fluid levels have been associated with many patients admitted into the ICU, and fluid resuscitation is effective for treating patients with severe sepsis or septic shock.

Chloride is measured as the concentration of chloride ions present within the blood. Chloride measurements in the blood can be obtained by performing a chemistry laboratory test. Chloride plays a role in maintaining fluid levels within the body's cells. An imbalance in chloride can result in an imbalance in fluid levels. Disturbances in fluid levels have been associated with many patients admitted into the ICU, and fluid resuscitation is effective for treating patients with severe sepsis or septic shock.

Total carbon dioxide is the measure of carbon dioxide found in the blood. Total carbon dioxide can be measured in the blood using a chemistry laboratory test. Total carbon dioxide can be used to identify metabolic acidosis which is a function of the respiratory system. An imbalance in Total carbon dioxide can be linked with an irregularity in the respiratory system. Elevated Total carbon dioxide measurements have been linked to an increase in mortality for sepsis patients.

Sodium is measured as the concentration of sodium ions present in the blood. Sodium measurements in the blood can be obtained by performing a chemistry laboratory test. Sodium plays a role in maintaining fluid levels within the body's cells. An imbalance in sodium can result in an imbalance in fluid levels. Disturbances in fluid levels have been associated with many patients admitted into the ICU, and fluid resuscitation can be effective for treating patients with severe sepsis or septic shock.

Albumin is measured as the concentration of albumin present in the blood. Albumin measurements in the blood can be obtained by performing a chemistry laboratory test. Albumin is a protein that is synthesized by the liver that minimizes the capillary leak into the body's tissues. An irregularity in Albumin can indicate a problem with the liver or renal system. Serum albumin has been identified as a predictor of mortality for septic patients.

Bilirubin is measured as the concentration of bilirubin present in the blood. Bilirubin measurements in the blood can be obtained by performing a chemistry laboratory test. Bilirubin is a protein that is generated during the breakdown of red blood cells. Bilirubin is a waste product that is filtered out by the liver in healthy patients. Bilirubin is used in the assessment of the liver component in the SOFA Score calculation.

Glucose concentration is the concentration of glucose present in the blood. Glucose concentration can be measured by performing a chemistry laboratory test. In sepsis, there is reduced blood flow within the body. If the glucose is unable overcome the interstitial space to reach its target, it may contribute to hyperglycemia.

Calcium is measured as the concentration of calcium ions present in the blood. Calcium measurements in the blood can be obtained by performing a chemistry laboratory test. Calcium plays a role in maintaining fluid levels within the body's cells. An imbalance in calcium can result in an imbalance in fluid levels. Disturbances in fluid levels have been associated with many patients admitted into the ICU, and fluid resuscitation can be effective for treating patients with severe sepsis or septic shock.

Lactate is measured as the concentration of lactate present in the blood. Lactate can be measured by a laboratory test. Lactate (or lactic acid) is generated by tissues during states of hypoxia. The build of lactate within the blood can indicate a patient is under organ failure or shock. Lactate has emerged as a part of the septic shock label in the Sepsis-3 definition.

As used herein, lab-derived biomarkers are mathematically derived measurements that measure's a patient's phenotype for a biological phenomenon. Lab-derived biomarkers are measured by obtaining a sample(s), measuring one or multiple biological properties of interest, and generating an index or score that correlates to a biological phenomenon using a mathematical method. An example is Cytovale® IntelliSep®, which measures single-cell biophysical properties by applying controlled pressure to white blood cells and collecting high fidelity images. The biophysical properties measurements are aggregated and converted into the Intellisep Index® (ISI) which aids in the diagnosis of sepsis in patients with suspected or confirmed infection.

As used herein, sepsis specific biomarkers are biomarkers that have shown to have high clinical utility for diagnosing or treating patients at high risk of having or developing sepsis. Sepsis specific biomarkers do not necessarily only show clinical utility for sepsis and may show utility for multiple medical conditions in tandem with sepsis. Sepsis-specific biomarker concentrations may include procalcitonin, C-reactive protein, protein biomarkers (e.g., Angiopoietin-1, Angiopoietin-2, Cystatin C, D-Dimer, E-Selectin, Fractalkine, FLT3-Ligand, GCSF, GDF15, GMCSF, Granzyme-B, IFN-alpha, IFN-gamma, IL1-beta, IL1-RA, IL-2, IL-4, IL-6, IL-7, IL-8, IL-10, IL-15, IP-10, lactate dehydrogenase (LDH), lipopolysaccharide-binding protein (LBP), Leptin, MCP1, MIP1-alpha, MIP1-beta, MIP3-alpha, NGAL, Pancreatic Stone Protein, PDL-1, Pentraxin-3, Protein C, S100b, TGF-alpha, Thromodulin, Tissue Factor, TNF-alpha, TRAIL, TREM-1, Troponin, VCAM-1, and/or VEG-F), genomic biomarkers (e.g., SNPs in CD14, TLR1, TLR2, TLR4, TLR6, TNF-alpha, IL-6, factor V, factor XII, FER, MAN2A1), gene expression or transcriptomic biomarkers (e.g., CEACAM4, LAMP1, PLAC8, PLA2G7, CEACAM1, ZDHHC19, C9orf95, GNA15, BATE, C3AR1, KIAA1370, TGFBI, MTCH1, RPGRIP1, HLA-DPB1, Nuclear factor-kappa B (NF-kB), Heme oxygenase-1 (HO-1), FCGR2C, CRP, IL-6, IL-10, IL-12, TNF, LY6E, LAMP3, ISG15, USP18, RSAD2, IFI44L, NCOA7, CMPK2, IFI44, BATF2, GBP4, IFIH1, GMPR, CXCL10, IFI27, ISG15, CCL2, OAS3, P2RY14, GCH1, CD274, ID01, ARL4A, BPGM, TNFAIP3, TNFAIP6, IL1R1, PROK2, RGS1, CXCR6, RTP4, TLR3, FZD5, KCNJ2, IRAK2, RGS16, CXCL8, OLR1, CCRL2, SPP1, ST3GAL4, CST7, IL1R2, IL4R, GALM, CTLA4, SOCS1, BCL2L1, TGM2, SLC39A8, CA2, PNP, GATA1, SLC1A5, RGS16, CD44, PDGFC, CSF2RA, CXCL1, CSF3R, SOCS3, IL18R1, PIM1, LEPR, IL1B, CSF2RB, ITGB3, STAT1, FAS, EFNA1, IER3, EETS2, SLC2A3, BCL2A1, PFKFB3, CEBPB, BIRC2, ATF3, TUBB2A, G0S2, MERTK, BIK, QSOX1, PYGL, TGFA, P4HA2, LDHA, IRS2, TSPO, HGF, GADD45A, TGFBR3, CD38, PRF1, FASLG, TIMP3, ANKH, LGALS3, PPP2R5B, GPX1, BNIP3L, BCL2L1, KLF7, FOSL2, ADM, MXI1, SELENBP1, PGF, FOXO3, NEDD4L, SLC2A1, SIAH2, MMP9, CXCR3, CD8A, IFNG, CD8B, JAK2, CCL4, ICAM1, WARS1, KRT1, GPR65, DYRK3, ACHE, ARG1, LCN2, LTF, OLFM4, HLA-DMB, YKT6, PDE4B, TWISTNB, BTN2A2, ZBTB33, PSMB9, CAMK4, TMEM19, SLC12A7, TP53BP1, PLEKHO1, SLC25A22, FRS2, CD24, STX1A, KCNMB4, CRISP2, HTRA1, PPL, RHBDF2, ZCCHC4, DDX6, SENP5, RAPGEF1, DTX2, RELB, DYRK2, CCNB1IP1, TDRD9, ZAP70, ARL14EP, MDC1, and/or ADGRE3), or composite biomarker scores (e.g., SeptiCyte® LAB and SeptiCyte® RAPID, or Inflammatix® Tri Verity®).

C-reactive Protein (CRP) is the concentration of CRP within the blood. CRP measurements in the blood can be obtained by performing a chemistry laboratory test. CRP is a protein that is produced by the liver whose level rises due to inflammation. Studies have shown that CRP when combined with temperature, can increase the detection of infection.

Procalcitonin (PCT) is measured as the concentration of PCT present in the blood. PCT can be measured by a laboratory test. PCT is released by tissues into the bloodstream during the presence of inflammation. Bacterial infections have shown to have a large impact on PCT synthesis, as bacterial endotoxins and cytokines facilitate host cells' PCT secretion.

The authentication module 110 manages authentication and authorization of users of the computing device 100, or of software executed by the computing device 100. For example, the measurements and input features of the patient are protected health information under the Health Insurance Portability and Accountability Act of 1996 (HIPAA), and therefore only authorized individuals can view such measurement and input features of the patient. The authentication module 110 is a measure that helps prevent unauthorized individuals from gaining access to the protected health information.

The pre-processing module 112 transforms input features that are received by the computing device 100 in order to match the parameters (e.g., units, scale, method, etc.) of the input features with which the machine learning model 120 was trained. In some instances, an input feature received by the computing device 100 may already match the parameters of the corresponding input feature on which the machine learning model 120 was trained and therefore does not require a transformation by the pre-processing module 112. In various embodiments, the pre-processing module 112 maps received input features by Logical Observation Identifiers Names and Codes (LOINC) code to the appropriate parameters of the corresponding input feature on which the machine learning model 120 was trained. A LOINC code, as is known to a person having skill in the art, is a universal standard for identifying medical laboratory observations that facilitates the exchange and pooling of results such as laboratory tests or vital signs for clinical care, outcomes management, and research. Mappings may determine any transformation functions on those received input features, such as unit or method conversions. Pre-processing and mapping configuration rules can be set by a user of the computing device 100. An example pre-processing procedure will be described in detail below.

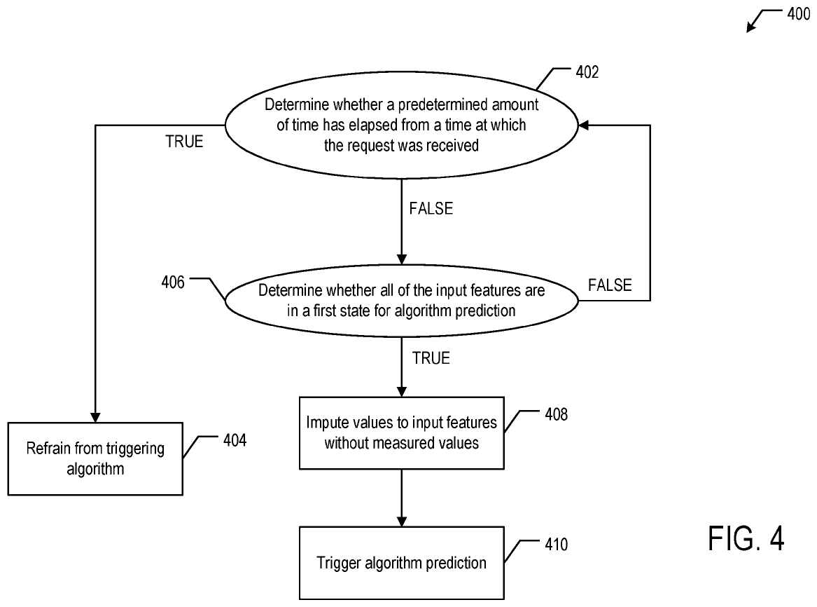

The trigger logic module 114 determines if and when to trigger the machine learning model 120 to generate a prediction. Example methods detailing how the trigger logic module 114 makes such a determination will be described in greater detail below under the subheading “Triggering Method” and in connection with FIGS. 4 to 6.

Model drift of the machine learning model 120 may be monitored indirectly by monitoring for data drift via the data drift module 116. The data drift module 116 performs tests to identify the possible occurrence of data drift of the machine learning model 120. In at least some embodiments, the data drift module 116 may generate data drift reports that show the results of the tests used. If at least one of the tests is failed, the data drift report indicates that data drift is detected. An end-user may then use the data drift report to determine whether data drift has truly occurred, and if so, to determine corrective action to take. Further details regarding the data drift detection process will be described below.

In some examples other than the depicted example, the components of the computer network 10 may be combined, rearranged, or removed, or intermediary components may be added. For example, the measurement device 102 and the user input device 104 may be combined. In another example, a measurement device 102 may transmit information to a computing device in a patient's room, which then transmits that information to the computing device 100. In another example, as stated above, the machine learning model 120 may be stored on a server in communication with the computing device over the network 106.

### Prediction Method

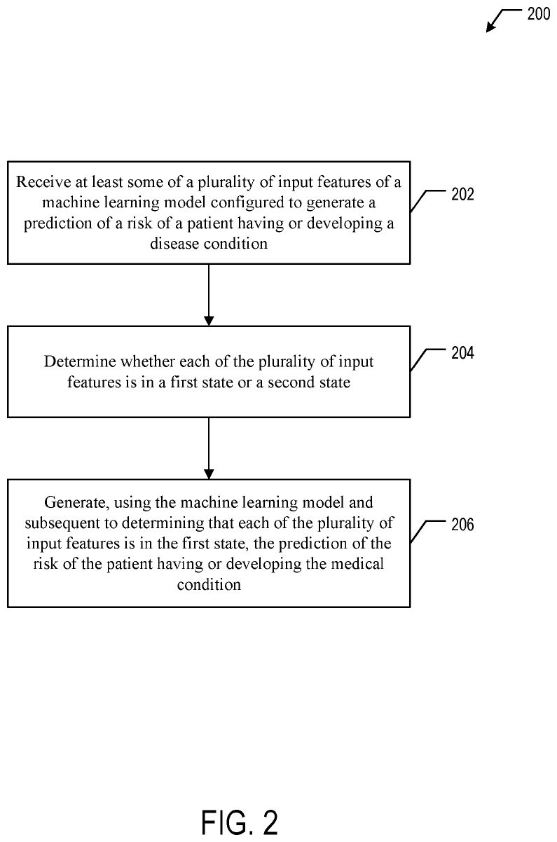

FIG. 2 is a flow chart of an example method 200 for generating a prediction of a risk level (e.g., probability) of a patient having or developing a medical condition (e.g., sepsis). The risk level, in various embodiments, may be a measure of risk for a predetermined amount of time (e.g., 24 hours) from the time at which a request is made to trigger an algorithm (e.g., machine learning model 120) to generate a prediction. Stated differently, in such embodiments, a generated prediction can no longer be relied upon after the predetermined amount of time elapses. The method 200 may be performed by processing logic that may comprise hardware (circuitry, dedicated logic, etc.), software, or a combination of both. For example, the computing device 100 may perform the method 200. In at least some embodiments, the method 200 may include authentication and authorization procedures to help prevent unauthorized individuals from gaining access to health information of a patient protected under HIPAA. The authentication and authorization procedures are managed through the configuration of users, access policies, and authentication clients. The authentication procedures may be executed prior to enabling a user to submit a request to trigger the machine learning model 120 or may be executed in response to receiving the request prior to moving forward in the method 200.

At block 202, a plurality of input features of a machine learning model (e.g., the machine learning model 120) built to generate a prediction of a risk of a patient having or developing a medical condition (e.g., sepsis) are received. For example, if the machine learning model 120 has twenty-two input features, then 1, 2, 3, 4, 5, 6, 7, 8, 9, 10, 11, 12, 13, 14, 15, 16, 17, 18, 19, 20, or 22 input features may be received. As will be described more below, input features that are not received at block 202 may be received at a later point in time or may be imputed with a substitute value at a later point in time. In at least some aspects, the received input features are values measured by one of the measurement devices 102 or values that are measured via clinical observation and input into one of the user input devices 104.

In at least some embodiments, the input features are received in real-time. Stated differently, in these embodiments, an input feature collected by the measurement device 102 or user input device 104 is transmitted to the computing device 100 instantaneously, or near instantaneously, as the input feature is collected (e.g., measured) such that the computing device 100 receives the input feature in real-time. The computing device 100 may store the input feature received in real-time in the patient data 124 stored in the memory 118.

In at least some embodiments, a request is received (e.g., by the processor 122) to trigger the machine learning model 120 to begin the process of generating the prediction of the risk of the patient having or developing the medical condition. In at least some embodiments, upon receiving the request (e.g., from a clinician's input device) to trigger the machine learning model 120, the processor 122 begins listening for input features from a variety of measurement devices 102 or user input devices 104, such as by reading the patient data 124 stored in the memory 118. In these embodiments, prior to the request being received, the processor 122 does not read the patient data 124. The patient data 124 can be continually updated as new information is received from the measurement devices 102 or user input devices 104 even though the processor 122 is not reading the patient data 124. Stated differently, in such embodiments, the computing device 100 continually receives and stores input features in the patient data 124, but the processor 122 does not begin execution of the method 200 to read the patient data 124 until the request is received. Once the processor 122 begins executing the method 200, the stored input features of the patient data 124 are read until either the machine learning model 120 generates an output or it is determined that the machine learning model 120 is unable to generate a prediction based on the available input features. In other embodiments, the processor 122 may continually read the stored input features regardless of a request. In such other embodiments, the processor 122 may wait to execute the method 200 beyond reading the stored patient data 124 until the request is received.

In various embodiments, receiving the request includes receiving a data packet that includes the following information: a time at which the request was received, a plurality of input features of the machine learning model 120 stored in the patient data 124 if available, a time at which an input feature was ordered for each of the input features if available, a time at which an input feature was collected for each of the input features if available, and a time at which an input feature resulted for each of the input features if available.

In various embodiments, the method 200 may include one or more pre-processing procedures for transforming one or more of the received input features to match a set of parameters of the input features on which the machine learning model 120 was trained (e.g., via the pre-processing module 112). Example transformations include: mapping laboratory and/or clinical observations (e.g., LOINC) to an input feature, unit conversion, log transformations, normalization of input features, standardization of input features, scaling, conversion of continuous measurements to categorical measurements (and vice versa), linear or non-linear mapping functions, The pre-processing procedures may include mapping the received input features to one or more parameters of the input features on which the machine learning model 120 was trained. For example, a transformation function may convert the unit or scale or method of a received input feature to match the unit or scale or method with which the machine learning model 120 was trained. In one example, the C-reactive protein input feature is commonly recorded using LOINC code 1988-5, and commonly measured using a machine (e.g., Roche Cobas® 6000 Analyzer Series Module c501) that outputs units of mg/L. In this example, a transformation function is needed to transform the measured C-reactive protein input feature to units of pg/ml, and transform the input feature value to a C-reactive protein measurement as measured on a Human Obesity Luminex Performance Assay Panel, because these are the units and method on which the machine learning model 120 was trained. In another example, an age of a patient may be converted into an age category (e.g., 65 and over, or under 65).

In another example, a transformation function is used for the procalcitonin input feature to convert the input feature measured using a machine (e.g., Roche Cobas® 6000 Analyzer Series Module c501) from units of ng/L to pg/ml and to transform the input feature to a Luminex Performance Human Premixed Multi-Analyte Panel Set 3C method.

Users (e.g., administrators) of the provided system (e.g., computing device 100) can configure the mappings for a particular organization and/or can configure normal ranges for each input feature for the organization. Based on the configured normal ranges, outliers outside of the minimum and maximum of the range may be identified and removed. When input features are displayed to an end-user, if any of the input features are outside the configured normal range, then those input features are indicated as such.

At block 204, a state for triggering the machine learning model is determined for each of the plurality of input features. For example, an input feature is in a first state indicative of the input feature being ready for algorithm prediction if the input feature is associated with a measured value that was measured within an acceptable time frame, or if the input feature is imputable and sufficient time as passed such that the input feature can be imputed with a substitute value. Each input feature of the input features has a respective set of requirements to be in the first state. In another example, an input feature is in a second state indicative of the input feature being not ready for algorithm prediction if the input feature is not associated with a measured value that was measured within an acceptable time frame and is not imputable, or if the input feature is imputable but sufficient time has not passed such that the input feature cannot be imputed with a substitute value.

For example, a set of requirements for a first input feature of the plurality of input features may require the first input feature to be associated with a first measured value that is resulted and acceptable in order for the first input feature to be in the first state because the first input feature has significant importance to the prediction of the machine learning model 120 and an imputed substitute value would diminish the accuracy of the prediction. As such, in this example, the first input feature is not imputable and will be in the second state unless a measured value is received for the first input feature. In some instances, after the first input feature is determined to be in the second state, the method 200 may include waiting a predetermined amount of time to receive a measured value for the first input feature. A state of the first input feature may be determined periodically during the predetermined amount of time. If a measured value for the first input feature is received at a later time within the predetermined amount of time, then the first input feature transitions to the first state. If a measured value for the first input feature is never received after the predetermined amount of time, then, in various aspects, the machine learning model 120 is not triggered because the first input feature remains in the second state. In other aspects,

In another example, a set of requirements of a second input feature of the plurality of input features are different because the second input feature is imputable and therefore not required to be associated with a resulted and acceptable measured value to be in the first state. When a measured value is not received for the second input feature (e.g., the second input feature is not received), the method 200 may include waiting a predetermined amount of time to receive the second input feature. The second input feature is in the second state, and therefore cannot be imputed with a substitute value, until the predetermined amount of time elapses because a measured value is preferred, even if not required, for the accuracy of the prediction of the machine learning model 120. Once the predetermined amount of time elapses, the second input feature is determined to be in the first state and can be imputed with a substitute value. As such, the set of requirements for the second input feature to be in the first state include that the second input feature is imputable and that a predetermined amount of time as elapsed.

Additional detail regarding determining a state of an input feature is provided under the subheading “Triggering Method” in connection with FIGS. 4 to 7 and their corresponding description.

At block 206, using the machine learning model 120, the prediction of the risk (e.g., probability) of the patient having or developing the medical condition (e.g., sepsis) is generated. In some embodiments, the prediction is generated subsequent to determining that each of the plurality of input features is in the first state such that the prediction is based on all of the plurality of input features of the machine learning model 120. In such embodiments, the prediction is not generated if one or more input features is in the second state, and the output of the machine learning model 120 is “no result”. Refraining from triggering the machine learning model 120 to generate a prediction until all input features have available values in these embodiments, and only imputing a value for certain input features in certain instances, results in consistently meaningful (e.g., accurate) outputs from the algorithm. In other embodiments, the machine learning model 120 may be triggered when one or more input features that cannot be imputed are missing. For instance, it may be predetermined, or determined as part of the method 200, that a certain combination of input features, or of input features and imputed input features, will generate a consistently meaningful (e.g., accurate) prediction without the one or more missing input features. In various examples, the prediction of the risk is a value between 0 and 100, though other suitable scales can be used. The prediction may be displayed on a display device (e.g., a display of the user input device 104).

In some embodiments, the generated prediction of the risk of the patient may be categorized into a risk category. For example, the predicted risk may be categorized into a first risk category (e.g., low risk), a second risk category (e.g., medium risk), a third risk category (e.g., high risk), or a fourth risk category (e.g., very high risk) based on thresholds defining each category. If the predicted risk is less than a first threshold, the predicted risk is categorized into the first risk category. In an example, the first risk category is based on a proportion of prior patients in the first risk category that required vasopressors within 24 hours relative to prior patients in the second, third, and fourth risk categories. If the predicted risk is greater than the first threshold but less than a second threshold, the predicted risk is categorized into the second risk category. In an example, the second risk category is based on a proportion of prior patients in the second risk category that required mechanical ventilation within 24 hours relative to prior patients in the first, third, and fourth risk categories. If the predicted risk is greater than the second threshold but less than a third threshold (e.g., the 95^{th} percentile of predicted risk), the predicted risk is categorized into the third risk category. In an example, the third risk category is based on a proportion of prior patients in the third risk category that required transfer to an intensive care unit within 24 hours relative to prior patients in the first, second, and fourth risk categories. If the predicted risk is greater than the third threshold, the predicted risk is categorized into the fourth risk category. In an example, the fourth risk category is based on a proportion of prior patients in the fourth risk category whose condition resulted in an in-hospital mortality relative to prior patients in the first, second, and third categories.

In some embodiments, at least some of the input features of the machine learning model 120 may include a local feature importance value, which is a numerical value associated with an input feature that signifies the input feature's contribution to the prediction of the machine learning model 120. For example, an input feature may include a Shapley Additive Explanations (SHAP) value, a Local interpretable model-agnostic explanations (LIME) value, or another suitable local feature importance value. In an example, the input features may include a local feature importance value for each input feature that is to be associated with an imputed value. The use of the local feature importance values aid in the interpretation and trust of the generated prediction. The local feature importance values for a single prediction when added equal to an uncalibrated prediction-the mean (uncalibrated prediction) across the pre-defined dataset. The additive impact of each input feature on the uncalibrated prediction can be observed. The local feature importance values are not a global machine learning model property, but rather a property of any new generated prediction, and localize the commonly used concept of feature importance for an individual prediction.

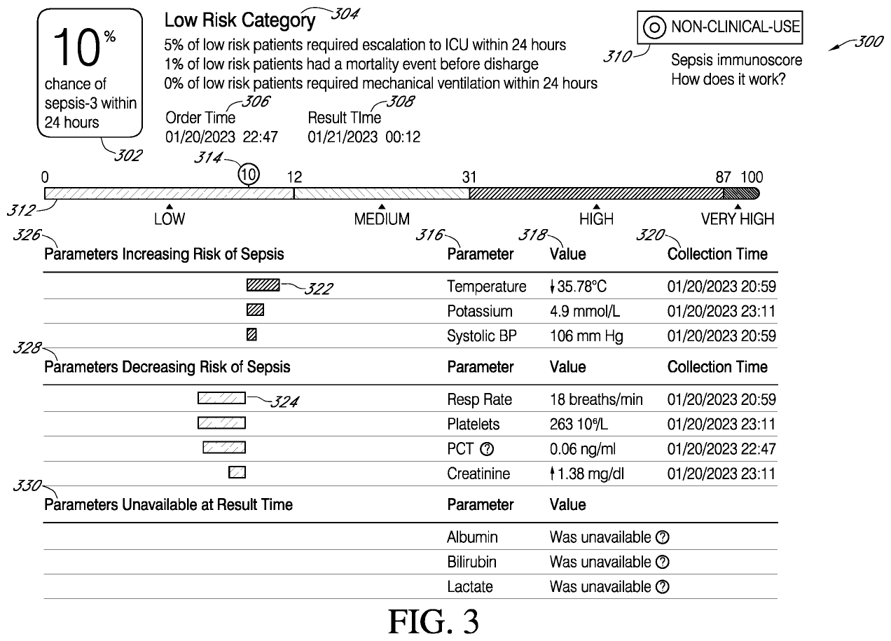

An example process to generate the local feature importance value contributions of the input features includes using the imputed input features, the trained machine learning model 120, and a training data object. Calculations of local feature importance values may use a game-theoretic approach to identify the contribution an input feature plays in the prediction of an observation. The local feature importance values use the training data object to identify estimations of the input feature contribution to the training dataset. Due to the computational complexity of calculating local feature importance values, in various embodiments, the process may use a local feature importance value estimation implementation via the fastshap R package. The fastshap R package may use Monte-Carlo simulations with 10 rounds to estimate the input feature contribution of the training data object with a fixed seed. This estimation can be applied to new observations to generate an input features' overall contribution (e.g., local feature importance value) to a prediction. A local feature importance value of an input feature can be visually represented on a display for the benefit of a clinician (e.g., see FIG. 3).

In at least some embodiments, the method 200 may include calibrating the prediction output by the machine learning model 120. For example, classifiers generated by random forest models, despite being both accurate and achieving a high AUC, produce class probabilities that are of rather poor quality, as measured by the squared error of the predicted probabilities (Brier score). One approach to addressing this problem is by means of a calibration model, such as learning a function that maps the original probability estimates, or scores, into more accurate probability estimates. For example, in various embodiments, a calibration model may be generated by performing a Platt calibration. A Platt calibration can be created by training a logistic regression model with the uncalibrated prediction of the machine learning model 120 to predict a calibrated prediction (e.g., a Sepsis-3 training label). Other suitable methods for generating the calibration model may be used in various embodiments, such as an isotonic calibration. The calibration model is a logistic model that uses the uncalibrated prediction of the machine learning model 120 as an input to generate a calibrated prediction. The purpose of the calibration model is to establish a relationship between the uncalibrated prediction and the classification of having or developing sepsis within 24 hours of the request to trigger the machine learning model 120. This relationship can be applied to convert future uncalibrated predictions into calibrated predictions. In such calibration embodiments, the calibrated prediction may be categorized into a risk category.

FIG. 3 illustrates an example user interface 300 that may be displayed (e.g., on a user input device 104) for an end-user (e.g., clinician) to assess the output of the machine learning model 120. The user interface 300 includes a predicted risk level 302 of a patient having or developing sepsis within the predetermined amount of time (e.g., 24 hours) from the time at which the request for the prediction was made. In this example, the patient's risk level (e.g., probability) of developing sepsis is 10% within 24 hours from the time at which the request was made. The user interface 300 further includes a risk category 304, which is a “low risk category” in this example. An order time 306 of when a request to trigger the machine learning model 120 to generate a prediction was received, and a result time 308 of when the prediction was generated are included. In various aspects, the computing device 100 may be operated in a “clinical use” or “non-clinical use” mode. If operated in “clinical use”, all outputs of the machine learning model 120 are labeled as “for clinical use” to the end-user healthcare provider. If operated in “non-clinical use”, all outputs of the machine learning model 120 are labeled as “for non-clinical use” to the end-user healthcare provider, indicating to an end-user healthcare provider that the result is not intended to be used for informing care of real patients. This labeling of results helps ensure patient safety when the device is being used outside of normal clinical use, such as, for example, during installation or testing of the computing device 100. In this example, the user interface 300 shows that the mode 310 of the computing device 100 was “non-clinical use.” A scale 312 may be shown that visually displays the different risk categories. In this example, the scale 312 is a bar, which may include different colors for each risk category. An indicator 314 is arranged on the scale 312 to visually display the patient's risk level in comparison to the risk categories.

Input parameters 316 to the machine learning model 120 are displayed in the bottom portion of the user interface 300. For each input parameter 316, a measured value 318 of the input parameter 316, if available, and a collection time 320 of the measured value 318, if available, are shown on the user interface 300. The input parameters 316 are categorized into parameters 326 that increase the patient's risk of developing sepsis, parameters 328 that decrease the patient's risk of developing sepsis, and parameters 330 that were unavailable at the time the prediction was generated. The parameters 330 are indicated as unavailable, in this example, in that they did not have a measured value 318 that could be used to generate the prediction, but rather had a value imputed to them. In other examples, the parameters 330 with imputed values may be displayed with their imputed values and with an indication that the values are imputed and not measured. For each input parameter 316 having a measured value 318, a bar is shown that indicates a contribution (e.g., local feature importance value) of an input parameter 316 to the patient's risk of developing sepsis. For example, each input parameter 316 of the parameters 326 is associated with a bar 322 that indicates a contribution (e.g., local feature importance value) of an input parameter 316 to increasing the patient's risk of developing sepsis. The larger the bar 322 is, the greater the contribution. In another example, each input parameter 316 of the parameters 328 is associated with a bar 324 that indicates a contribution (e.g., local feature importance value) of an input parameter 316 to decreasing the patient's risk of developing sepsis. The larger the bar 324 is, the greater the contribution.

In some embodiments, the method 200 includes detecting model drift indirectly by detecting data drift of the machine learning model 120. Detecting data drift may include comparing data input into the processing logic directing the method 200 (e.g., software) during an “observed” period to a “reference” period. The reference period is intended to select data that is representative of future observations. The reference period can be modified by an authenticated end-user if needed. The observed period is specific to a particular data drift report. All input data input into the machine learning model 120 during the specified period is considered a part of the observed dataset. The comparison process performs several statistical tests to check for differences in data distributions and data availability. In at least some embodiments, a data drift report may be generated that communicates the results of the statistical tests. A detected difference could be an indication that data drift has occurred. In various aspects, if at least one of the statistical tests is failed, the data drift report indicates that the data drift is detected for the observed data set. If a difference is detected, or a data drift report indicates that data drift is detected, an end-user can assess whether data drift has occurred, take steps to protect patient safety, if necessary, and correct the data drift problem, if necessary.

In various embodiments, there are two categories of input data that are monitored for data drift: measurement results and measurement orders. Measurement results are measurements that have a result/recorded time and a value. Measurement results serve as inputs to the machine learning model. Measurement results will be monitored for changes in distribution and availability. Measurement orders are un-resulted measurements. Measurement orders are not directly used as inputs to the machine learning model 120, but aid in determining when the machine learning model 120 will be triggered. Measurement orders cannot be monitored for changes in distribution since no values are associated with them. However, measurement orders can be monitored for changes in availability. In some aspects, measurement results and measurement orders will only be used for data drift reports if the measurement results and measurement orders fall within a time window surrounding the request to trigger the machine learning model 120. In an example, the time window is from the beginning of the staleness period for the measurement type, until the result time of the prediction generated, or lack thereof, by the machine learning model 120.

A first example statistical test is used to detect changes in measurement result distributions. In this first example, using a Kolmogorov-Smirnov test, each measurement value distribution is compared between the reference set and the observed set. Based on a specified p-value (e.g., 0.05), if the null hypothesis of the Two-Sample Kolmogorov-Smirnov test is rejected, this signifies that the reference set and observed set do not stem from the same distribution for the parameter, which indicates that there may be a change in the distribution of measurement values between the reference and observed set.

A second example statistical test is used to detect changes in measurement result availability. In this second example, for each request to trigger the machine learning model 120 and each measurement type, the frequency of measurement results available is calculated by dividing the number of measurements available by the length of a time window associated with a respective request to trigger the machine learning model 120. The calculation will be performed for both the reference and observed set. Using a Two-Sample Kolmogorov-Smirnov test in this second example, the distribution of measurement availability frequencies is compared between the reference and observed set. Based on a specified p-value (e.g., 0.05), if the null hypothesis of the Two-Sample Kolmogorov-Smirnov test is rejected, this signifies that the reference set and observed set do not stem from the same distribution for the parameter, which indicates that there may be a difference in the availability of measurement orders between the reference and observed set.

A third example statistical test is used to detect changes in measurement ordering availability. In this third example, for each request to trigger the machine learning model 120 and each measurement type, the frequency of measurement orders is calculated by dividing the number of measurement orders by the length of a time window associated with a respective request to trigger the machine learning model 120. The calculation will be performed for both the reference and observed set. Using a Two-Sample Kolmogorov-Smirnov test in this second example, the distribution of measurement ordering frequencies is compared between the reference and observed set. Based on a specified p-value (e.g., 0.05), if the null hypothesis of the Two-Sample Kolmogorov-Smirnov test is rejected, this signifies that the reference set and observed set do not stem from the same distribution for the parameter, which indicates that there may be a difference in the measurement ordering between the reference and observed set.

### Triggering Method

FIG. 4 shows a flow chart of an example method 400 for determining whether to trigger an algorithm (e.g., the machine learning model 120) stored on a computing device (e.g., the computing device 100). The method 400 may be performed by processing logic that may comprise hardware (circuitry, dedicated logic, etc.), software, or a combination of both. At oval 402, it is determined whether a predetermined amount of time (e.g., 3.5 hours) has elapsed from a time at which the request to trigger the algorithm was received. This predetermined amount of time at oval 402 sets the maximum time limit for waiting for input features before the algorithm cannot be triggered and a new request to trigger the algorithm will have to be made. If the predetermined amount of time has not elapsed (e.g., FALSE), then, at oval 406, it is determined whether each of a plurality of input features to the algorithm are in the first state such that the plurality of input features are ready (e.g., available) for algorithm prediction. In some aspects, the plurality of input features are all of the input features of the algorithm. In other aspects, the plurality of input features are a subset of all of the input features of the algorithm. For instance, it may be predetermined, or determined as part the method 400, that a certain combination of input features (some of which may be imputed) less than all of the input features of the algorithm will generate an accurate prediction without the one or more missing input features.

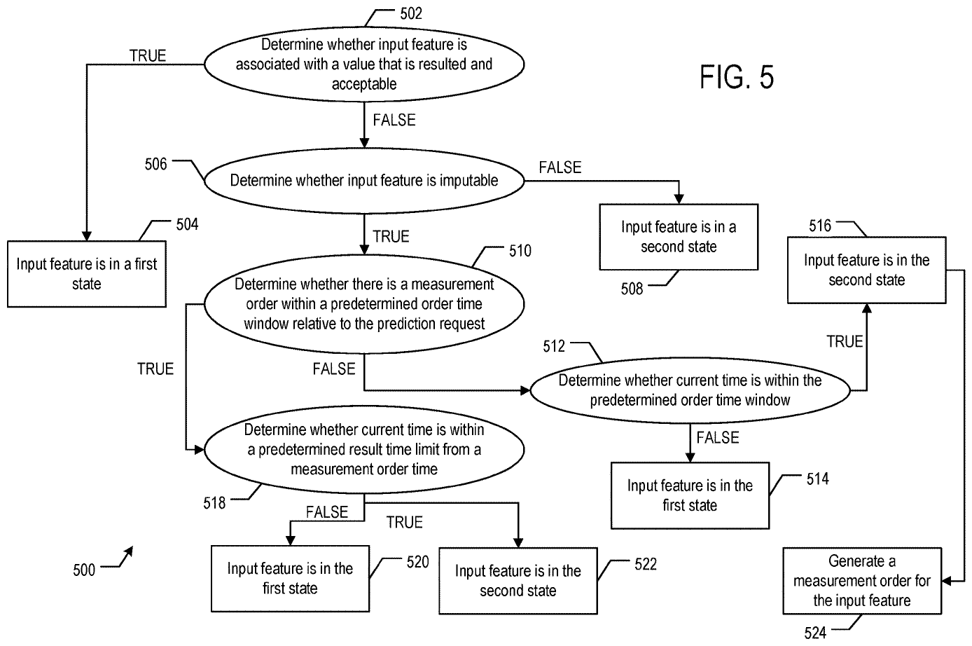

FIG. 5 shows a flow chart of an example method 500 for determining whether an input feature for an algorithm stored on a computing device is in the first state such that the input feature is ready (e.g., available) for algorithm prediction. The method 500 may be performed for each of the plurality of input features of the algorithm in order to determine, at oval 406, whether each of the plurality of input features meet their respective set of requirements to be in the first state. The method 500 may be performed by processing logic that may comprise hardware (circuitry, dedicated logic, etc.), software, or a combination of both. At oval 502, it is determined whether an input feature is resulted and acceptable. A input feature is resulted when the input feature is measured and received (e.g., from a measurement device 102). A input feature is acceptable when the input feature was measured (e.g., collected from a patient) within a predetermined amount of time from a time at which the request to trigger the algorithm was received (e.g., acceptable time window) such that the input feature is likely to still be accurate. For instance, measured values from a comprehensive metabolic panel are not likely to be an accurate representation of a patient's current condition if the blood tests for the panel results were run four days prior. As such, the input features from this four-day-old comprehensive metabolic panel are not acceptable because they would negatively affect the accuracy of the algorithm prediction.

How long a measured input feature remains valid, i.e. the predetermined amount of time from the time at which the request to trigger the algorithm was received, can vary for different input features. For example, a patient's age changes only once a year whereas the patient's blood composition could change over the course of a day. In an example, the following measured input features remain valid for six hours: systolic blood pressure, diastolic blood pressure, temperature, respiratory rate, heart rate, blood oxygen saturation, and, if included as input features, fraction of inspired oxygen (FiO2) and Glasgow coma scale. In another example, the following measured input features remain valid for twenty-four hours: white blood cell count, lymphocyte count, neutrophil count, platelet count, creatinine, blood urea nitrogen, potassium, chloride, total carbon dioxide, sodium, albumin, bilirubin, procalcitonin, C-reactive protein, lactate, and, if included as input features, glucose, calcium, hematocrit, and hemoglobin. Additionally, a measurement time of an input feature can vary for different input features. For example, a measurement time for laboratory measurements may correspond to a draw time (e.g., time blood is drawn from a patient) whereas a measurement time for patient vitals may correspond to a recording time of those vitals.

In some aspects, more than one instance of an input feature may be measured and acceptable value. For example, a temperature sensor can collect a patient's temperature every few seconds thereby resulting in many measured temperature input features that are within the acceptable time window for the temperature input feature. In such instances, a single measured input feature within the acceptable time window is selected. The single measured value can be selected from the multiple values in any suitable manner (e.g., minimum, maximum, average, most recent, etc.) and can vary for different input features. For example, a minimum measured instance of an input feature can be selected for the following input features: systolic blood pressure, diastolic blood pressure, and blood oxygen saturation. In another example, a maximum measured instance of an input feature can be selected for the following input features: temperature, respiratory rate, and heart rate. In another example, a measured instance of an input feature most recent in time can be selected for the following input features: white blood cell count, lymphocyte count, neutrophil count, platelet count, creatinine, blood urea nitrogen, potassium, chloride, total carbon dioxide, sodium, albumin, bilirubin, procalcitonin, C-reactive protein, and lactate.

If it is determined at oval 502 that the measured input feature is resulted and acceptable, then, at block 504, the input feature is determined to be ready (e.g., available) for algorithm prediction (e.g., in the first state). In some instances, the set of requirements of an input feature to be in the first state may include the input feature being resulted and acceptable. If instead it is determined at oval 502 that the input feature is either not resulted or is not acceptable, then, at oval 506, it is determined whether the input feature is imputable. In some embodiments, to determine whether an input feature is imputable a lookup table may be consulted that lists each of the input features and whether each is imputable (e.g., YES or NO). In other embodiments, determining whether an input feature is imputable may depend on other input features. For instance, the imputability of an input feature may depend on whether one or more other input features have resulted and acceptable values. In an example, C-reactive protein may be imputable if procalcitonin (PCT), albumin, and platelet count have resulted and acceptable values, but otherwise C-reactive protein is not imputable. In another instance, the imputability of an input feature may additionally or alternatively depend on the resulted and acceptable value of one or more other input features (e.g., being within a same range, meeting a threshold, failing to meet a threshold, etc.). In an example, C-reactive protein may be imputable if albumin has a resulted and acceptable value, and procalcitonin (PCT) has a resulted and acceptable value less than 2log(10) pg/mL, but otherwise C-reactive protein is not imputable.

In various embodiments, the following input features may be imputable: age, lymphocyte count, neutrophil count, potassium, chloride, total carbon dioxide, sodium, albumin, bilirubin, lactate, and, if included as input features, fraction of inspired oxygen (FiO2), Glasgow coma scale, glucose, calcium, hematocrit, and hemoglobin concentration. In various embodiments, the following input features are not imputable: systolic blood pressure, diastolic blood pressure, blood oxygen saturation, temperature, respiratory rate, heart rate, white blood cell count, platelet count, creatinine, blood urea nitrogen, procalcitonin, and C-reactive protein.

If the input feature is not imputable, then, at block 508, it is determined that the input feature is not ready for algorithm prediction (e.g., in a second state). In this instance, the set of requirements of the input feature to be in the first state include the input feature being resulted and acceptable. In some embodiments, the method 500 may terminate for this input feature after determining that the input feature is not ready. In other embodiments, at block 524, it is determined whether a measurement order for the input feature has been placed within a predetermined amount of time (e.g., predetermined order time window) relative to the time of the received request to trigger the algorithm (e.g., block 202). The predetermined order time window can capture a period of time after the received trigger request and/or before the received trigger request. A measurement order being placed indicates that a measured input feature is considered informative (e.g., by the ordering clinician, hospital policies, etc.) and should therefore be leveraged if possible. A time limit is set, however, for the measurement order to be placed in order to balance the benefit of the informative measured input feature and the need for a prompt algorithm prediction. The order time window intends to generate a timely imputation of a substitute value for an input feature if a physician has no intention of ordering a measurement of the input feature while giving the opportunity for the physician to order the measurement of the input feature by not imputing too quickly. In an example, the predetermined order time window is three hours before an algorithm trigger request and thirty minutes after the trigger request for each of the input features. In some aspects, the predetermined order time window may be different between different input features. If a measurement order has been placed, then the input feature remains in the second state at block 508 and the method 500 terminates.

If it is instead determined at block 524 that there is not a measurement order within the predetermined order time window, then, at block 526, an active ordering method is triggered. FIG. 5B shows a flow chart of an example active ordering method 550 that may be performed at block 526. The active ordering method 550 is a feature that can be turned on or off for specific input features. As such, at block 552, it is determined whether active ordering is enabled for the input feature. For example, a lookup table may be consulted that lists each of the input features and whether active order is enabled for each input feature (e.g., YES or NO). If active ordering is not enabled for an input feature, then the input feature remains in the second state at block 554 and method 500 terminates.

If active ordering is enabled for the input feature, then, at block 556, it is determined whether automatic feature measurement ordering is enabled for the input feature. For example, a lookup table may be consulted that lists each of the input features and whether automatic feature measurement ordering is enabled for each input feature (e.g., YES or NO). If automatic feature measurement ordering is not enabled, then, at block 558, a notification to prompt a user to generate a measurement order may be generated for the input feature. In some aspects of the active ordering method 500, a notification that additional parameters should be obtained for the patient in order for the machine learning model 120 to have the highest chance of triggering in a timely and accurate fashion may be communicated to a clinician (e.g., via a display of the clinician's user input device 104) so that the clinician can generate a measurement order as timely as possible. In some instances, the notification that additional parameters should be obtained is, additionally or alternatively, added to a patient's hospital electronic medical record.

If instead automatic feature measurement ordering is enabled for the input feature, then, at block 560, a measurement order for the input feature may be automatically generated and transmitted (e.g., by computing device 100 executing the active ordering method), such as to a lab technician's user input device 104 or a measurement device 102, rather than relying upon a clinician to generate the measurement order. Automatically generating a measurement order may include translating an input feature for which the measurement order is generated to a measurement order. The automatically generated measurement order may instruct a machine (e.g., a measurement device 102) or healthcare professional (e.g., lab technician) to conduct a laboratory test or vital assessment. As such, this active ordering method may actively alert a clinician or communicate with a hospital electronic medical record, user input device 104, and/or measurement device 102 to trigger prompt ordering of necessary labs or patient evaluations to provide necessary data to the machine learning model 120 in as timely a fashion as possible. The laboratory test or vital assessment may be configured by administrators of the system 10.

At block 562, the active ordering method 500 may involve processing logic executing the active order method 500 communicating with processing logic executing the triggering method to indicate that an input feature is expected to be available in a timely fashion. For instance, the active ordering logic may communicate a time that a measured input feature is expected based on a measurement order being placed, either by a clinician (or other medical professional) or the computing device 100. In some aspects, communication from the active ordering logic may cause the trigger logic to adjust the order time window and/or the result time limit for waiting for the missing input features before resulting. For example, if the predetermined order time window is supposed to expire in 15 minutes, but the active ordering logic communicates that a missing measured input feature is expected to result in 25 minutes, then the trigger logic may extend the order time window (e.g., so that it expires in 30 minutes) so that the measured input feature can be used in the prediction determination instead of an imputed input feature or refraining from the prediction entirely which causes the prediction process to start from the beginning. A similar time extension can be executed for the predetermined result time limit. In some aspects, at least one of the order time window and the result time limit may be extended more than once. For example, continuing with the example above, if after 29 minutes, the active ordering logic communicates to the trigger logic that the measured value is expected to result in 3 minutes (i.e. after the extended order time window), then the trigger logic may extend the order time window again, such as for another 5 or 10 minutes. The trigger logic may extend at least one of the order time window and the result time limit any suitable amount of time (e.g., 5, 10, 15, 20, 25, 30, 35, 40, 45, 50, 55, 60, or 120 minutes, etc.) relative to how much time will have elapsed after the prediction request if the result time limit is extended, the remaining time for each of the resulted input features to remain acceptable, an impact, on the prediction, of a particular input feature being imputed rather than having a resulted and acceptable value, and other suitable considerations. The trigger logic may also refrain from extending at least one of the order time window and the result time limit based on the same above considerations for extending at least one of the order time window and the result time limit. Once the order time window and/or the result time limit are updated if needed, the method 500 terminates.

Returning to oval 510 of FIG. 5A, if instead the input feature is imputable, then it is determined whether a measurement order has been placed within a predetermined amount of time (e.g., predetermined order time window) relative to the time of the received request to trigger the algorithm (i.e. block 202). Determining whether the measurement order has been placed may be executed in the same manner, and has the same benefits, as described above for block 524.

If there is not a measurement order placed within the predetermined order time window, then, at oval 512, it is determined whether the current time is within the predetermined order time window. Stated differently, it is determined whether the time window for placing a measurement order has finished or not. If it is determined that the current time is outside the predetermined order time window, then, at block 514, it is determined that the input feature is ready (e.g., in the first state). If instead it is determined that the current time is still within the predetermined order time window, then, at block 516, it is determined that the input feature is not ready (e.g., in the second state). Stated differently, the input feature is not ready because time remains for a measurement order to be placed, and thus there remains the possibility that the missing resulted and acceptable input feature could be received rather than needing to impute a value to the input feature. In this instance, the set of requirements of the input feature to be in the first state include either: (1) being associated with a resulted and acceptable value or (2) being imputable and the predetermined order time window elapsing.

In some embodiments, the method 500 terminates after determining that the input feature is not ready at block 516. In other embodiments, the method 500 returns to block 526 to determine (e.g., in the same way as block 526 is described above) whether a measurement order for the input feature has been placed within the predetermined order time window relative to the time of the received request to trigger the algorithm (e.g., block 202). If a measurement order has been placed, then the input feature remains in the second state and the method 500 terminates for this input feature. If it is determined at block 524 that there is not a measurement order within the predetermined order time window, then, at block 526, a measurement order, or a notification to generate a measurement order, may be generated for the input feature as part of the active ordering logic described above.

Returning to oval 510, if there is a measurement order placed within the predetermined order time window, then, at oval 518, it is determined whether the current time is within a predetermined result time limit from a time at which the measurement order was placed. The result time limit allows an ordered measurement to be collected and resulted while still enabling a timely algorithm prediction if there is a major delay in the collection and measurement result process and a value for the input feature instead needs to be imputed. In this instance, the set of requirements of the input feature to be in the first state include either: (1) being associated with a resulted and acceptable value or (2) being imputable and the predetermined result time window elapsing. Examples of input features that may require time for a measured value after a measurement is ordered include laboratory tests, remotely observed phenomena that must be transmitted long distances, observations taken by devices that are not immediately accessible, and many others. In at least one example, the predetermined result time limit is two hours from the measurement order time for each of the input features. In some aspects, the predetermined result time limit may be different between different measurement orders associated with different input features. If it is determined that the current time is outside the predetermined result time limit, then, at block 520, it is determined that the input feature is ready (e.g., in the first state). If instead it is determined that the current time is still within the predetermined result time limit, then, at block 522, it is determined that the input feature is not ready (e.g., in the second state). Stated differently, the input feature is not ready because time remains for a measurement to be resulted, and thus there remains the possibility that the input feature could become associated with a resulted and acceptable value rather than needing to impute a value to the input feature.

Returning to oval 406 of FIG. 4, if an instance of the method 500 determines that one or more input feature of the plurality of input features are not ready for algorithm prediction (e.g., FALSE), then the method 400 returns to oval 402 to determine whether the predetermined amount of time has elapsed from when the request was received. In some aspects, the method 400 may include generating a notification communicating that a result of the algorithm prediction is still pending and displaying that notification on a display of a device (e.g., a user input device 104). If the predetermined amount of time has elapsed at oval 402, then, at block 404, the method 400 includes refraining from triggering the algorithm. Stated differently, if the predetermined amount of time elapses from the time at which the request was received without each of the plurality of input features being ready for algorithm prediction, then the method 400 ends without the algorithm being triggered. In some aspects, the method 400 may include generating a notification communicating that the algorithm cannot be triggered and displaying that notification on a display of a device (e.g., a user input device 104).

If instead the predetermined amount of time has still not elapsed at oval 402, then the method 400 can again proceed to oval 406 to determine whether all of the input features are now ready for algorithm prediction. For instance, a measured input feature resulting from a measurement order for the input feature may have resulted since the previous time it was determined whether each input feature was ready. This loop from oval 406 to oval 402 can repeat as many times as needed. If each of the plurality of input features are ready for algorithm prediction (e.g., TRUE), then, at block 408, substitute values are imputed to the input features that are not resulted and acceptable (e.g., block 502).

Determining a suitable substitute value to impute for an input feature can be carried out in a variety of suitable manners. In some aspects, a substitute value can be predetermined for an input feature such that, if the input feature needs an imputed value, then the substitute value is imputed to the input feature. For example, in the context of the example algorithm for predicting a patient's risk of having or getting sepsis, a value of 21% may be imputed to the fraction of inspired oxygen (FiO2) input feature when imputation is necessary because 21% is the value of FiO2 for someone breathing air at sea level, and in the absence of evidence that a patient is receiving oxygen support, this value is a reasonable assumption.

In other aspects, a substitute value for imputing to a particular input feature can be calculated using a statistical or machine learning model (e.g., that may be stored in the memory 118 or on a remote computing device in communication with the computing device 100. In such aspects, a supervised machine learning model (e.g., random forest model) is trained with the imputable input feature as the label (i.e., outcome) and the remainder of the input features of the algorithm are features (i.e., covariates, predictors) in the model. For example, if the plurality of input features of the algorithm has four input features and a substitute value must be calculated for the first input feature, then the other three input features are inputs to the machine learning model to generate the substitute value. The machine learning model may be trained using any suitable software. For example, the bag imputation method in the R caret package, which uses bootstrap aggregating (i.e. bagging) regression trees as the supervised learning models, may be used. In some aspects, predetermined substitute values may be imputed to some input features while values determined based on a model may be imputed to other input features.

Once each of the plurality of input features is either resulted and acceptable or imputed with a substitute value, the algorithm is triggered to generate a prediction, at block 210, based on the input features and imputed input features.

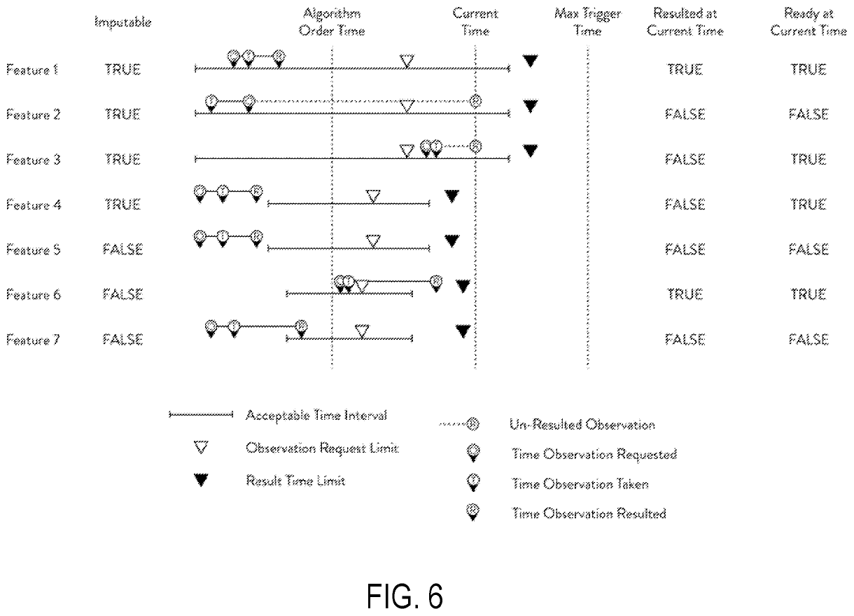

FIG. 6 illustrates a chart of example state determinations for seven input features based on the methods 400 and 500 detailed above. As shown, Feature 1 has a resulted observation (e.g., measured value) that was taken (e.g., measured) within an acceptable time interval and is therefore determined to be ready (e.g., in the first state). Feature 2 does not have a resulted value, but is imputable and does have an observation request (e.g., measurement order) that was made subsequent to the starting point of the acceptable time interval and prior to the observation request limit (e.g., within the predetermined order time window). The current time is still prior to the result time limit so Feature 2 is determined to be not ready (e.g., in the second state). Feature 3 does not have a resulted value though is imputable. Feature 3 does not have a measurement order within the predetermined order time window because the measurement order was placed after the predetermined order time window, and the current time is after the predetermined order time window. Therefore Feature 3 is determined to be in the first state. Feature 4 has a resulted value though it was measured outside the acceptable time interval and is therefore not acceptable. Feature 4 is imputable, does not have a measurement order that is un-resulted, and the current time is after the predetermined order time window. Therefore Feature 4 is determined to be in the first state.

Feature 5 has a resulted value though it was measured outside the acceptable time interval and is therefore not acceptable. Feature 5 is not imputable and therefore is determined to be in the second state. Feature 6 has a resulted value that was measured within the acceptable time interval and is therefore determined to be in the first state. Feature 7 has a resulted value that resulted within the acceptable time interval; however, the resulted value was measured (e.g., time observation taken) outside the acceptable time interval and therefore the resulted value is not acceptable. Feature 7 is not imputable and is therefore determined to be in the second state.

### Computer-Implemented System

FIG. 7 is a block diagram that illustrates a computer system 700, upon which embodiments of the present teachings may be implemented. In various embodiments of the present teachings, computer system 700 can include a bus 702 or other communication mechanism for communicating information, and a processor 704 coupled with bus 702 for processing information. In various embodiments, computer system 700 can also include a memory, which can be a random access memory (RAM) 706 or other dynamic storage device, coupled to bus 702 for determining instructions to be executed by processor 704. Memory also can be used for storing temporary variables or other intermediate information during execution of instructions to be executed by processor 704. In various embodiments, computer system 700 can further include a read only memory (ROM) 708 or other static storage device coupled to bus 702 for storing static information and instructions for processor 704. A storage device 710, such as a magnetic disk or optical disk, can be provided and coupled to bus 702 for storing information and instructions.

In various embodiments, computer system 700 can be coupled via bus 702 to a display 712, such as a cathode ray tube (CRT) or liquid crystal display (LCD), for displaying information to a computer user. An input device 714, including alphanumeric and other keys, can be coupled to bus 702 for communicating information and command selections to processor 704. Another type of user input device is a cursor control 716, such as a mouse, a trackball or cursor direction keys for communicating direction information and command selections to processor 704 and for controlling cursor movement on display 712. This input device 714 typically has two degrees of freedom in two axes, a first axis (i.e., x) and a second axis (i.e., y), that allows the device to specify positions in a plane. However, it should be understood that input devices 714 allowing for 3 dimensional (x, y and z) cursor movement are also contemplated herein.

Consistent with certain implementations of the present teachings, results can be provided by computer system 700 in response to processor 704 executing one or more sequences of one or more instructions contained in memory 706. Such instructions can be read into memory 706 from another computer-readable medium or computer-readable storage medium, such as storage device 710. Execution of the sequences of instructions contained in memory 706 can cause processor 704 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 704 for execution. Such a medium can take many forms, including but not limited to, non-volatile media, volatile media, and transmission media. Examples of non-volatile media can include, but are not limited to, optical, solid state, magnetic disks, such as storage device 710. Examples of volatile media can include, but are not limited to, dynamic memory, such as memory 706. Examples of transmission media can include, but are not limited to, coaxial cables, copper wire, and fiber optics, including the wires that comprise bus 702.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 704 of computer system 700 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein via flow charts, diagrams and accompanying disclosure can be implemented using computer system 700 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Rust, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 700, whereby processor 704 would execute the analyses and determinations provided by these engines, subject to instructions provided by any one of, or a combination of, memory components 706/708/710 and user input provided via input device 714.

### Digital Processing Device

In various embodiments, the systems and methods described herein can include a digital processing device, or use of the same. In various embodiments, the digital processing device can includes one or more hardware central processing units (CPUs) or general-purpose graphics processing units (GPGPUs) that carry out the device's functions. In various embodiments, the digital processing device further comprises an operating system configured to perform executable instructions. In various embodiments, the digital processing device can be optionally connected a computer network. In various embodiments, the digital processing device can be optionally connected to the Internet such that it accesses the World Wide Web. In various embodiments, the digital processing device can be optionally connected to a cloud computing infrastructure. In various embodiments, the digital processing device can be optionally connected to an intranet. In various embodiments, the digital processing device can be optionally connected to a data storage device.

In accordance with various embodiments, suitable digital processing devices can include, by way of non-limiting examples, server computers, desktop computers, laptop computers, notebook computers, sub-notebook computers, netbook computers, netpad computers, handheld computers, Internet appliances, mobile smartphones, tablet computers, and personal digital assistants. Those of ordinary skill in the art will recognize that many smartphones are suitable for use in the system described herein. Those of ordinary skill in the art will also recognize that select televisions, video players, and digital music players with optional computer network connectivity are suitable for use in the system described herein. Suitable tablet computers include those with booklet, slate, and convertible configurations, known to those of ordinary skill in the art.

In various embodiments, the digital processing device includes an operating system configured to perform executable instructions. The operating system can be, for example, software, including programs and data, which manages the device's hardware and provides services for execution of applications. Those of ordinary skill in the art will recognize that suitable server operating systems include, by way of non-limiting examples, FreeBSD, OpenBSD, Net-BSD, Linux, Apple® Mac OS X Server®, Oracle® Solaris®, Windows Server®, and Novell® NetWare®. Those of ordinary skill in the art will recognize that suitable personal computer operating systems include, by way of non-limiting examples, Microsoft® Windows®, Apple® Mac OS X®, UNIX®, and UNIX-like operating systems such as GNU/Linux®. In various embodiments, the operating system is provided by cloud computing. Those of ordinary skill in the art will also recognize that suitable mobile smart phone operating systems include, by way of non-limiting examples, Nokia® Symbian® OS, Apple® iOS®, Research In Motion® Black-Berry OS®, Google® Android®, Microsoft® Windows Phone® OS, Microsoft® Windows Mobile® OS, Linux®, and Palm® WebOS®.

In various embodiments, the device includes a storage and/or memory device. The storage and/or memory device is one or more physical apparatuses used to store data or programs on a temporary or permanent basis. In various embodiments, the device is volatile memory and requires power to maintain stored information. In various embodiments, the device is non-volatile memory and retains stored information when the digital processing device is not powered. In various embodiments, the non-volatile memory comprises flash memory. In some embodiments, the non-volatile memory comprises dynamic random-access memory (DRAM). In various embodiments, the non-volatile memory comprises ferroelectric random access memory (FRAM). In various embodiments, the non-volatile memory comprises phase-change random access memory (PRAM). In various embodiments, the device is a storage device including, by way of non-limiting examples, CD-ROMs, DVDs, flash memory devices, magnetic disk drives, magnetic tapes drives, optical disk drives, and cloud computing based storage. In various embodiments, the storage and/or memory device is a combination of devices such as those disclosed herein.

In various embodiments, the digital processing device includes a display to send visual information to a user. In various embodiments, the display is a cathode ray tube (CRT). In various embodiments, the display is a liquid crystal display (LCD). In various embodiments, the display is a thin film transistor liquid crystal display (TFT-LCD). In various embodiments, the display is an organic light emitting diode (OLED) display. In various embodiments, on OLED display is a passive-matrix OLED (PMOLED) or active-matrix OLED (AMOLED) display. In various embodiments, the display is a plasma display. In various embodiments, the display is a video projector. In various embodiments, the display is a combination of devices such as those disclosed herein.

In various embodiments, the digital processing device includes an input device to receive information from a user. In various embodiments, the input device is a keyboard. In various embodiments, the input device is a pointing device including, by way of non-limiting examples, a mouse, trackball, track pad, joystick, game controller, or stylus. In various embodiments, the input device is a touch screen or a multi-touch screen. In various embodiments, the input device is a microphone to capture voice or other sound input. In various embodiments, the input device is a video camera or other sensor to capture motion or visual input. In various embodiments, the input device is a Kinect, Leap Motion, or the like. In various embodiments, the input device is a combination of devices such as those disclosed herein.

### Non-Transitory Computer Readable Storage Medium

In various embodiments, and as stated above, the systems and methods disclosed herein can include, and the methods herein can be run on, one or more non-transitory computer readable storage media encoded with a program including instructions executable by the operating system of an optionally networked digital processing device. In various embodiments, a computer readable storage medium is a tangible component of a digital processing device. In various embodiments, a computer readable storage medium is optionally removable from a digital processing device. In various embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In various embodiments, the program and instructions are permanently, substantially permanently, semi-permanently, or non-transitorily encoded on the media.

### Computer Program

In various embodiments, the systems and methods disclosed herein can include at least one computer program, or use at least one computer program. A computer program includes a sequence of instructions, executable in the digital processing device's CPU, written to perform a specified task. Computer readable instructions may be implemented as program modules, such as functions, objects, Application Programming Interfaces (APis), data structures, and the like, that perform particular tasks or implement particular abstract data types. Those of ordinary skill in the art will recognize that a computer program may be written in various versions of various languages.

The functionality of the computer readable instructions may be combined or distributed as desired in various environments. In various embodiments, a computer program comprises one sequence of instructions. In various embodiments, a computer program comprises a plurality of sequences of instructions. In various embodiments, a computer program is provided from one location. In various embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In various embodiments, a computer program includes a web application. Those of ordinary skill in the art will recognize that a web application, in various embodiments, utilizes one or more software frameworks and one or more database systems. In various embodiments, a web application is created upon a software framework such as Microsoft® .NET or Ruby on Rails (RoR). In various embodiments, a web application utilizes one or more database systems including, by way of non-limiting examples, relational, non-relational, object oriented, associative, and XML database systems. In various embodiments, suitable relational database systems include, by way of non-limiting examples, Microsoft® SQL Server, mySQL™, and Oracle®. Those of ordinary skill in the art will also recognize that a web application, in various embodiments, is written in one or more versions of one or more languages. A web application may be written in one or more markup languages, presentation definition languages, client-side scripting languages, server-side coding languages, data-base query languages, or combinations thereof. In various embodiments, a web application is written to some extent in a markup language such as Hypertext Markup Language (HTML), Extensible Hypertext Markup Language (XHTML), or extensible Markup Language (XML). In various embodiments, a web application is written to some extent in a presentation definition language such as Cascading Style Sheets (CSS). In various embodiments, a web application is written to some extent in a client-side scripting language such as Asynchronous Javascript and XML (AJAX), Flash® ActionScript, JavaScript, or Silverlight®. In various embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In various embodiments, a web application is written to some extent in a database query language such as Structured Query Language (SQL). In various embodiments, a web application integrates enterprise server products such as IBM® Lotus Domino®. In various embodiments, a web application includes a media player element. In various embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In various embodiments, a computer program includes a mobile application provided to a mobile digital processing device. In various embodiments, the mobile application is provided to a mobile digital processing device at the time it is manufactured. In various embodiments, the mobile application is provided to a mobile digital processing device via the computer network described herein.

A mobile application can be created by techniques known to those of ordinary skill in the art using hardware, languages, and development environments known to the art. Those of ordinary skill in the art will recognize that mobile applications can be written in several languages. Suitable programming languages include, by way of non-limiting examples, C, C++, C#, Objective-C, Java™, JavaScript, Pascal, Object Pascal, Rust, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelera-tor®, Celsius, Bedrock, Flash Lite, .NET Compact Frame-work, Rhomobile, and WorkLight Mobile Platform. Other development environments are available without cost including, by way of non-limiting examples, Lazarus, Mobi-Flex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of ordinary skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nin-tendo DSi Shop.

### Standalone Application

In various embodiments, a computer program includes a standalone application, which is a program that is run as an independent computer process, not an add-on to an existing process, e.g., not a plug-in. Those of ordinary skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In various embodiments, a computer program includes one or more executable complied applications.

### Web Browser Plug-In

In various embodiments, the computer program includes a web browser plug-in (e.g., extension, etc.). In computing, a plug-in is one or more software components that add specific functionality to a larger software application. Makers of software applications support plug-ins to enable third-party developers to create abilities, which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of ordinary skill in the art will be familiar with several web browser plug-ins including, Adobe® Flash® Player, Microsoft® Silver-light®, and Apple® QuickTime®. In various embodiments, the toolbar comprises one or more web browser extensions, add-ins, or add-ons. In various embodiments, the toolbar comprises one or more explorer bars, tool bands, or desk bands.

Those of ordinary skill in the art will recognize that several plug-in frame works are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Fire-fox®, Google® Chrome, Apple® Safari®, Opera Soft-ware® Opera®, and KDE Konqueror. In various embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

### Software Modules

In various embodiments, the systems and methods disclosed herein include a software, server and/or database modules, or incorporate use of the same in methods according to various embodiments disclosed herein. Software modules can be created by techniques known to those of ordinary skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In various embodiments, software modules are in one computer program or application. In various embodiments, software modules are in more than one computer program or application. In various embodiments, software modules are hosted on one machine. In various embodiments, software modules are hosted on more than one machine. In various embodiments, software modules are hosted on cloud computing platforms. In various embodiments, software modules are hosted on one or more machines in one location. In various embodiments, software modules are hosted on one or more machines in more than one location.

### Databases

In various embodiments, the systems and methods disclosed herein include one or more databases, or incorporate use of the same in methods according to various embodiments disclosed herein. Those of ordinary skill in the art will recognize that many databases are suitable for storage and retrieval of user, query, token, and result information. In various embodiments, suitable databases include, by way of non-limiting examples, relational databases, non-relational databases, object oriented databases, object databases, entity-relation-ship model databases, associative databases, and XML databases. Further non-limiting examples include SQL, Postgr-eSQL, MySQL, Oracle, DB2, and Sybase. In various embodiments, a database is internet-based. In further Web. Suitable web browsers include, by way of non-limiting examples, Microsoft® Internet Explorer®, Mozilla® Fire-fox®, Google® Chrome, Apple® Safari®, Opera Soft-ware® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

In various embodiments, a database is web-based. In various embodiments, a database is cloud computing-based. In other embodiments, a database is based on one or more local computer storage devices.

### Data Security

In various embodiments, the systems and methods disclosed herein include one or features to prevent unauthorized access. The security measures can, for example, secure a user's data. In various embodiments, data is encrypted. In various embodiments, access to the system requires multi-factor authentication and access control layer. In various embodiments, access to the system requires two-step authentication (e.g., web-based interface). In various embodiments, two-step authentication requires a user to input an access code sent to a user's e-mail or cell phone in addition to a username and password. In some instances, a user is locked out of an account after failing to input a proper username and password. The systems and methods disclosed herein can, in various embodiments, also include a mechanism for protecting the anonymity of users' genomes and of their searches across any genomes.

### Additional Considerations

Any headers and/or subheaders between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability, or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the embodiments claimed herein.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context. The phrase “and/or” means and or or. To illustrate, A, B, and/or C includes: A alone, B alone, C alone, a combination of A and B, a combination of A and C, a combination of B and C, or a combination of A, B, and C. In other words, “and/or” operates as an inclusive or.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

### Embodiments

1. A computer-implemented method for predicting a risk of a patient having or developing a medical condition, comprising:
receiving, by the processor, a plurality of input features of a machine learning model configured to generate a prediction of the risk of the patient having or developing the medical condition;
determining, by the processor, whether each of the plurality of input features is in a first state or a second state, wherein the first state and the second state are indicative of whether an input feature of the plurality of input features is available for triggering the machine learning model, and wherein each of the plurality of input features has a respective set of requirements to be in the first state; and
if every input feature in the plurality of input features is in a first state, generating, by the processor using the machine learning model, the prediction of the risk of the patient having or developing the medical condition.

2. The computer-implemented method of embodiment 1, wherein a first input feature of the plurality of input features is required to be associated with a first measured value in order for the first input feature to be in the first state.

3. The computer-implemented method of embodiment 2, further comprising refraining, by the processor, from triggering the machine learning model in response to determining, by the processor, that the first input feature is in the second state.

4. The computer-implemented method of embodiment 3, wherein the at least some of the plurality of input features are received at a first time point, the method further comprising receiving, by the processor, the first input feature at a second time point subsequent to the first time point.

5. The computer-implemented method of embodiment 1, further comprising:
determining, by the processor, that a first input feature of the plurality of input features is in the second state;
determining, by the processor, whether a measurement order for the first input feature has been generated subsequent to determining that the first input feature is in the second state; and
generating, by the processor and in response to determining that the measurement order for the first input feature has not been generated, at least one of (1) a notification alerting that the measurement order for the first input feature is needed or (2) the measurement order for the first input feature.

6. The computer-implemented method of any of embodiments 1 to 5, further comprising imputing, by the processor, a substitute value to a second input feature of the plurality of input features prior to generating the prediction using the machine learning model.

7. The computer-implemented method of embodiment 6, wherein the substitute value associated with the second input feature is determined, by the processor, using a second machine learning model, and wherein a second plurality of input features to the second machine learning model used to calculate the substitute value are the plurality of input features except for the second input feature.

8. The computer-implemented method of embodiment 6, wherein the prediction is automatically generated using the machine learning model upon imputing the substitute value to the second input feature.

9. The computer-implemented method of any of embodiments 1 to 8, further comprising transforming, by the processor, a third input feature such that the third input feature after transformation matches a set of parameters with which the machine learning model was trained.

10. The computer-implemented method of any of embodiments embodiment 1 to 9, further comprising detecting, by the processor, data drift of the machine learning model.

11. The computer-implemented method of any of embodiments 1 to 10, further comprising receiving, by the processor, a request to trigger the machine learning model at a first time point, wherein a fourth input feature is received at a second time point, and wherein determining whether each of the plurality of input features is in the first state comprises:
determining, by the processor at a third time point, a state of the fourth input feature, wherein the state of the fourth input feature is determined to be in the first state when the second time point is within a first predetermined time window, and wherein the state of the first input feature is determined to be in the second state when the second time point is outside the first predetermined time window.

12. The computer-implemented method of embodiment 11, wherein the first predetermined time window includes a period of time prior to the first time point.

13. The computer-implemented method of any of embodiments 1 to 10, further comprising receiving, by the processor, a request to trigger the machine learning model at a first time point, wherein the machine learning model generates the prediction based in part on a fifth input feature of the plurality of input features, and wherein determining whether each of the plurality of input features is in the first state comprises:
determining, by the processor at a second time point, a state of the fifth input feature, wherein determining the state of the fifth input feature includes:
   determining, by the processor, that the fifth input feature is imputable,
   determining, by the processor, whether an order has been initiated within a second predetermined time window for a measured value of the fifth input feature, wherein
      if there has been no order initiated and the second time point is subsequent to the second predetermined time window, then the fifth input feature is in the first state, and
      if there has been no order initiated and the second time point is within the second predetermined time window, then the fifth input feature is in the second state,
   determining, by the processor and in response to determining that the order has been initiated, whether the second time point is within a third predetermined time window, wherein
      if the second time point is within the third predetermined time window, then the fifth input feature is in the second state,
      if the second time point is subsequent to the third predetermined time window, then the fifth input feature is in the first state;
determining, by the processor and in response to determining that the fifth input feature is in the first state, a substitute measurement value for the fifth input feature; and imputing, by the processor, the substitute measurement value to the fifth input feature.

14. The computer-implemented method of embodiment 13, further comprising:
determining, by the processor, that the fifth input feature is in the second state;
determining, in response to determining that the fifth input feature is in the second state, that a predetermined amount of time has not yet elapsed since the first time point; and
determining, at a third time point, a state of the fifth input feature.

15. The computer-implemented method of embodiment 13, wherein the third predetermined time window begins at the second time point.

16. The computer-implemented method of any of embodiments 1 to 15, wherein the plurality of input features includes at least one input feature of a first group, at least one input feature of a second group, and at least one input feature of a third group,
wherein the first group includes the input features in the group consisting of: age, sex at birth, race, ethnicity, past medical history of the patient, current complaints or symptoms of the patient, neurological assessments, clinical decision support alerts, clinician or other chart notes, diagnosis codes, procedures performed on the patient, current medications of the patient, interventions, patient care setting, medical imaging data or assessments, electrograms, endoscopic tests, systolic blood pressure, diastolic blood pressure, body temperature, respiratory rate, heart rate, blood oxygen saturation, and a fraction of inspired oxygen,
wherein the second group includes the input features in the group consisting of: white blood cell count, lymphocyte count, neutrophil count, platelet count, creatinine, blood urea nitrogen, potassium, chloride, total carbon dioxide, sodium, albumin, bilirubin, lactate, glucose, calcium, hematocrit, hemoglobin concentration, lab-derived biomarkers, clinical severity measures and composites, biopsies data, and microorganism test data, and
wherein the third group includes the input features in the group consisting of: procalcitonin, C-reactive protein, protein biomarkers, genomic biomarkers, gene expression or transcriptomic biomarkers, and composite biomarker scores.

17. The computer-implemented method of embodiment 16, wherein the plurality of input features include:
age, diastolic blood pressure, heart rate, pulse oximetry, respiratory rate, systolic blood pressure, and temperature from the first group,
white blood cell count, lymphocyte count, neutrophil count, platelet count, creatinine, blood urea nitrogen, potassium, chloride, total carbon dioxide, sodium, albumin, bilirubin, and lactate from the second group, and
procalcitonin and C-reactive protein from the third group.

18. The computer-implemented method of any of embodiments 1 to 17, further comprising receiving, by the processor, a request to trigger the machine learning model to generate the prediction, wherein the processor begins reading patient data stored in a memory upon receiving the request, wherein the patient data includes the at least some of the plurality of input features, and wherein the at least some of the plurality of input features are received in real-time as the at least some of the plurality of input features are measured.

19. The computer-implemented method of any of embodiments 1 to 18, wherein the prediction further includes a category of the prediction and an importance value of a sixth input feature of the plurality of input features, wherein the importance value is indicative of a contribution of the sixth input feature to the prediction.

20. The computer-implemented method of any of embodiments 1 to 19, wherein the at least some of the plurality of input features includes a plurality of seventh input features, the method further comprising selecting, by the processor, a single seventh input feature of the plurality of seventh input features with which to generate the prediction.

21. The computer-implemented method of any of embodiments 1 to 20, further comprising:
receiving, by the processor, an indication that a measured value of a first input feature of the plurality of input features is expected to be received at a first time outside of a predetermined time window; and
adjusting, by the processor, the predetermined time window to generate an adjusted time window, wherein the first time is within the adjusted time window.

22. The computer-implemented method of any of embodiments 1 to 21, wherein the plurality of input features are all of the input features of the machine learning model.

23. A system comprising:
a memory; and
a processor in communication with the memory, the processor configured to perform operations including:
   receiving a plurality of input features of a machine learning model configured to generate a prediction of the risk of the patient having or developing the medical condition;
   determining whether each of the plurality of input features is in a first state or a second state, wherein the first state and the second state are indicative of whether an input feature of the plurality of input features is available for triggering the machine learning model, and wherein each of the plurality of input features has a respective set of requirements to be in the first state; and
   if every input feature in the plurality of input features is in a first state, generating, using the machine learning model, the prediction of the risk of the patient having or developing the medical condition.

24. The system of embodiment 23, wherein a first input feature of the plurality of input features is required to be associated with a first measured value in order for the first input feature to be in the first state.

25. The system of embodiment 24, wherein the operations further include refraining from triggering the machine learning model in response to determining that the first input feature is in the second state.

26. The system of embodiment 25, wherein the operations further include:
determining that a first input feature of the plurality of input features is in the second state;
determining whether a measurement order for the first input feature has been generated subsequent to determining that the first input feature is in the second state; and
automatically generating the order for the first input feature and in response to determining that the measurement order for the first input feature has not been generated, at least one of (1) a notification alerting that the measurement order for the first input feature is needed or (2) the measurement order for the first input feature.

27. The system of any of embodiment 25, wherein the at least some of the plurality of input features are received at a first time point, the method further comprising receiving the first input feature at a second time point subsequent to the first time point.

28. The system of any of embodiments 23 to 27, wherein the operations further include imputing a substitute value to a second input feature of the plurality of input features prior to generating the prediction using the machine learning model.

29. The system of embodiment 28, wherein the substitute value associated with the second input feature is determined, using a second machine learning model, and wherein a second plurality of input features to the second machine learning model used to calculate the substitute value are the plurality of input features except for the second input feature.

30. The system of embodiment 28, wherein the prediction is automatically generated using the machine learning model upon imputing the substitute value to the second input feature.

31. The system of any of embodiments 23 to 30, wherein the operations further include transforming a third input feature such that the third input feature after transformation matches a set of parameters with which the machine learning model was trained.

32. The system of any of embodiments 23 to 31, wherein the operations further include detecting data drift of the machine learning model.

33. The system of any of embodiments 23 to 32, wherein the operations further include:
receiving, by the processor, a request to trigger the machine learning model at a first time point, wherein a fourth input feature is received at a second time point, and wherein determining whether each of the plurality of input features is in the first state comprises:
   determining, at a third time point, a state of the fourth input feature, wherein the state of the fourth input feature is determined to be in the first state when the second time point is within a first predetermined time window, and wherein the state of the first input feature is determined to be in the second state when the second time point is outside the first predetermined time window.

34. The system of embodiment 33, wherein the first predetermined time window includes a period of time prior to the first time point.

35. The system of any of embodiments 23 to 34, wherein the operations further include receiving a request to trigger the machine learning model at a first time point, wherein the machine learning model generates the prediction based in part on a fifth input feature of the plurality of input features, and wherein determining whether each of the plurality of input features is in the first state comprises:
determining, at a second time point, a state of the fifth input feature, wherein determining the state of the fifth input feature includes:
   determining that the fifth input feature is imputable,
   determining whether an order has been initiated within a second predetermined time window for a measured value of the fifth input feature, wherein
      if there has been no order initiated and the second time point is subsequent to the second predetermined time window, then the fifth input feature is in the first state, and
      if there has been no order initiated and the second time point is within the second predetermined time window, then the fifth input feature is in the second state,
   determining, in response to determining that the order has been initiated, whether the second time point is within a third predetermined time window, wherein
      if the second time point is within the third predetermined time window, then the fifth input feature is in the second state,
      if the second time point is subsequent to the third predetermined time window, then the fifth input feature is in the first state;
determining, in response to determining that the fifth input feature is in the first state, a substitute measurement value for the fifth input feature; and
imputing the substitute measurement value to the fifth input feature.

36. The system of embodiment 35, wherein the operations further include:
determining, by the processor, that the fifth input feature is in the second state;
determining, in response to determining that the fifth input feature is in the second state, that a predetermined amount of time has not yet elapsed since the first time point; and
determining, at a third time point, a state of the fifth input feature.

37. The system of embodiment 35, wherein the third predetermined time window begins at the second time point.

38. The system of any of embodiments 23 to 37, wherein the plurality of input features includes at least one input feature of a first group, at least one input feature of a second group, and at least one input feature of a third group,
wherein the first group includes the input features in the group consisting of: age, sex at birth, race, ethnicity, past medical history of the patient, current complaints or symptoms of the patient, neurological assessments, clinical decision support alerts, clinician or other chart notes, diagnosis codes, procedures performed on the patient, current medications of the patient, interventions, patient care setting, medical imaging data or assessments, electrograms, endoscopic tests, systolic blood pressure, diastolic blood pressure, body temperature, respiratory rate, heart rate, blood oxygen saturation, and a fraction of inspired oxygen,
wherein the second group includes the input features in the group consisting of: white blood cell count, lymphocyte count, neutrophil count, platelet count, creatinine, blood urea nitrogen, potassium, chloride, total carbon dioxide, sodium, albumin, bilirubin, lactate, glucose, calcium, hematocrit, hemoglobin concentration, lab-derived biomarkers, clinical severity measures and composites, biopsies data, and microorganism test data, and
wherein the third group includes the input features in the group consisting of: procalcitonin, C-reactive protein, protein biomarkers, genomic biomarkers, gene expression or transcriptomic biomarkers, and composite biomarker scores.

39. The system of embodiment 38, wherein the plurality of input features include:
age, diastolic blood pressure, heart rate, pulse oximetry, respiratory rate, systolic blood pressure, and temperature from the first group;
white blood cell count, lymphocyte count, neutrophil count, platelet count, creatinine, blood urea nitrogen, potassium, chloride, total carbon dioxide, sodium, albumin, bilirubin, and
lactate from the second group; and procalcitonin and C-reactive protein from the third group.

40. The system of any of embodiments 23 to 39, wherein the operations further include receiving a request to trigger the machine learning model to generate the prediction, wherein the processor begins reading patient data stored in a memory upon receiving the request, wherein the patient data includes the at least some of the plurality of input features, and wherein the at least some of the plurality of input features are received in real-time as the at least some of the plurality of input features are measured.

41. The system of any of embodiments 23 to 40, wherein the prediction further includes a category of the prediction and an importance value of a sixth input feature of the plurality of input features, wherein the importance value is indicative of a contribution of the sixth input feature to the prediction.

42. The system of any of embodiments 23 to 41, wherein the at least some of the plurality of input features includes a plurality of seventh input features, the operations further including selecting a single seventh input feature of the plurality of seventh input features with which to generate the prediction.

43. The system of any of embodiments 23 to 42, wherein the operations further include:
receiving an indication that a measured value of a first input feature of the plurality of input features is expected to be received at a first time outside of a predetermined time window; and
adjusting the predetermined time window to generate an adjusted time window, wherein the first time is within the adjusted time window.

44. The system of any of embodiments 23 to 43, wherein the plurality of input features are all of the input features of the machine learning model.

45. A non-transitory, computer-readable medium storing instructions, which when executed by a processor, cause the processor to perform operations including:
receiving a plurality of input features of a machine learning model configured to generate a prediction of the risk of the patient having or developing the medical condition;
determining whether each of the plurality of input features is in a first state or a second state, wherein the first state and the second state are indicative of whether an input feature of the plurality of input features is available for triggering the machine learning model, and wherein each of the plurality of input features has a respective set of requirements to be in the first state; and
if every input feature in the plurality of input features is in a first state, generating, using the machine learning model, the prediction of the risk of the patient having or developing the medical condition.

46. The non-transitory, computer-readable medium of embodiment 45, wherein a first input feature of the plurality of input features is required to be associated with a first measured value in order for the first input feature to be in the first state.

47. The non-transitory, computer-readable medium of embodiment 46, wherein the operations further include refraining from triggering the machine learning model in response to determining that the first input feature is in the second state.

48. The non-transitory, computer-readable medium of embodiment 47, wherein the operations further include:
determining that a first input feature of the plurality of input features is in the second state;
determining whether a measurement order for the first input feature has been generated subsequent to determining that the first input feature is in the second state; and
automatically generating the order for the first input feature and in response to determining that the measurement order for the first input feature has not been generated, at least one of (1) a notification alerting that the measurement order for the first input feature is needed or (2) the measurement order for the first input feature.

49. The non-transitory, computer-readable medium of embodiment 47, wherein the at least some of the plurality of input features are received at a first time point, the method further comprising receiving the first input feature at a second time point subsequent to the first time point.

50. The non-transitory, computer-readable medium of any of embodiments 45 to 49, wherein the operations further include imputing a substitute value to a second input feature of the plurality of input features prior to generating the prediction using the machine learning model.

51. The non-transitory, computer-readable medium of embodiment 50, wherein the substitute value associated with the second input feature is determined, using a second machine learning model, and wherein a second plurality of input features to the second machine learning model used to calculate the substitute value are the plurality of input features except for the second input feature.

52. The non-transitory, computer-readable medium of embodiment 50, wherein the prediction is automatically generated using the machine learning model upon imputing the substitute value to the second input feature.

53. The non-transitory, computer-readable medium of any of embodiments 45 to 52, wherein the operations further include transforming a third input feature such that the third input feature after transformation matches a set of parameters with which the machine learning model was trained.

54. The non-transitory, computer-readable medium of any of embodiments 45 to 53, wherein the operations further include detecting data drift of the machine learning model.

55. The non-transitory, computer-readable medium of any of embodiments 45 to 54, wherein the operations further include:
receiving, by the processor, a request to trigger the machine learning model at a first time point, wherein a fourth input feature is received at a second time point, and wherein determining whether each of the plurality of input features is in the first state comprises:
   determining, at a third time point, a state of the fourth input feature, wherein the state of the fourth input feature is determined to be in the first state when the second time point is within a first predetermined time window, and wherein the state of the first input feature is determined to be in the second state when the second time point is outside the first predetermined time window.

56. The non-transitory, computer-readable medium of embodiment 55, wherein the first predetermined time window includes a period of time prior to the first time point.

57. The non-transitory, computer-readable medium of any of embodiments 45 to 56, wherein the operations further include receiving a request to trigger the machine learning model at a first time point, wherein the machine learning model generates the prediction based in part on a fifth input feature of the plurality of input features, and wherein determining whether each of the plurality of input features is in the first state comprises:
determining, at a second time point, a state of the fifth input feature, wherein determining the state of the fifth input feature includes:
   determining that the fifth input feature is imputable,
   determining whether an order has been initiated within a second predetermined time window for a measured value of the fifth input feature, wherein
      if there has been no order initiated and the second time point is subsequent to the second predetermined time window, then the fifth input feature is in the first state, and
      if there has been no order initiated and the second time point is within the second predetermined time window, then the fifth input feature is in the second state,
   determining, in response to determining that the order has been initiated, whether the second time point is within a third predetermined time window, wherein
      if the second time point is within the third predetermined time window, then the fifth input feature is in the second state,
      if the second time point is subsequent to the third predetermined time window, then the fifth input feature is in the first state;
determining, in response to determining that the fifth input feature is in the first state, a substitute measurement value for the fifth input feature; and
imputing the substitute measurement value to the fifth input feature.

58. The non-transitory, computer-readable medium of embodiment 57, wherein the operations further include:
determining, by the processor, that the fifth input feature is in the second state;
determining, in response to determining that the fifth input feature is in the second state, that a predetermined amount of time has not yet elapsed since the first time point; and
determining, at a third time point, a state of the fifth input feature.

59. The non-transitory, computer-readable medium of embodiment 57, wherein the third predetermined time window begins at the second time point.

60. The non-transitory, computer-readable medium of any of embodiments 45 to 59, wherein the plurality of input features includes at least one input feature of a first group, at least one input feature of a second group, and at least one input feature of a third group,
wherein the first group includes the input features in the group consisting of: age, sex at birth, race, ethnicity, past medical history of the patient, current complaints or symptoms of the patient, neurological assessments, clinical decision support alerts, clinician or other chart notes, diagnosis codes, procedures performed on the patient, current medications of the patient, interventions, patient care setting, medical imaging data or assessments, electrograms, endoscopic tests, systolic blood pressure, diastolic blood pressure, body temperature, respiratory rate, heart rate, blood oxygen saturation, and a fraction of inspired oxygen,
wherein the second group includes the input features in the group consisting of: white blood cell count, lymphocyte count, neutrophil count, platelet count, creatinine, blood urea nitrogen, potassium, chloride, total carbon dioxide, sodium, albumin, bilirubin, lactate, glucose, calcium, hematocrit, hemoglobin concentration, lab-derived biomarkers, clinical severity measures and composites, biopsies data, and microorganism test data, and
wherein the third group includes the input features in the group consisting of: procalcitonin, C-reactive protein, protein biomarkers, genomic biomarkers, gene expression or transcriptomic biomarkers, and composite biomarker scores.

61. The non-transitory, computer-readable medium of embodiment 60, wherein the plurality of input features include:
age, diastolic blood pressure, heart rate, pulse oximetry, respiratory rate, systolic blood pressure, and temperature from the first group;
white blood cell count, lymphocyte count, neutrophil count, platelet count, creatinine, blood urea nitrogen, potassium, chloride, total carbon dioxide, sodium, albumin, bilirubin, and
lactate from the second group; and procalcitonin and C-reactive protein from the third group.

62. The non-transitory, computer-readable medium of any of embodiments 45 to 61, wherein the operations further include receiving a request to trigger the machine learning model to generate the prediction, wherein the processor begins reading patient data stored in a memory upon receiving the request, wherein the patient data includes the at least some of the plurality of input features, and wherein the at least some of the plurality of input features are received in real-time as the at least some of the plurality of input features are measured.

63. The non-transitory, computer-readable medium of any of embodiments 45 to 62, wherein the prediction further includes a category of the prediction and an importance value of a sixth input feature of the plurality of input features, wherein the importance value is indicative of a contribution of the sixth input feature to the prediction.

64. The non-transitory, computer-readable medium of any of embodiments 45 to 63, wherein the at least some of the plurality of input features includes a plurality of seventh input features, the method further comprising selecting, by the processor, a single seventh input feature of the plurality of seventh input features with which to generate the prediction.

65. The non-transitory, computer-readable medium of any of embodiments 45 to 64, wherein the operations further include:
receiving an indication that a measured value of a first input feature of the plurality of input features is expected to be received at a first time outside of a predetermined time window; and
adjusting the predetermined time window to generate an adjusted time window, wherein the first time is within the adjusted time window.

66. The non-transitory, computer-readable medium of any of embodiments 45 to 65, wherein the plurality of input features are all of the input features of the machine learning model.

1.-66. (canceled)

67. A computer-implemented method for predicting a risk of a patient having or developing a medical condition, comprising:
receiving, by a processor, clinical data associated with a plurality of input features of a machine learning model configured to generate a prediction of the risk of the patient having or developing the medical condition;
determining, by the processor, whether each of the plurality of input features is in a first state or a second state, wherein the first state and the second state are indicative of whether an input feature of the plurality of input features is available for triggering the machine learning model, and wherein each of the plurality of input features has a respective set of requirements to be in the first state; and
if every input feature in the plurality of input features is in a first state, generating, by the processor using the machine learning model, the prediction of the risk of the patient having or developing the medical condition.

68. The computer-implemented method of claim 67, wherein determining whether each input feature is in the first state or second state comprises evaluating, for each input feature:
(a) whether the input feature has a resulted observation taken within an acceptable time interval,
(b) whether the input feature is imputable based on an imputable flag,
(c) whether an observation request exists within an observation request limit relative to a current time,
(d) whether a result time limit has been exceeded from a time observation was requested, and/or
(e) whether a maximum trigger time has been exceeded from receipt of the request, wherein the first state requires either a resulted observation within the acceptable time interval or satisfaction of imputation criteria based on the imputable flag and associated time limits.

69. The computer-implemented method of claim 68, wherein determining the first state or second state for an input feature comprises: for non-imputable input features, requiring a resulted observation within the acceptable time interval, for imputable input features, determining the state based on a combination of: (i) presence or absence of an observation request within the observation request limit, (ii) elapsed time relative to the result time limit when an observation request exists, and (iii) current time relative to the observation request limit when no observation request exists, wherein different combinations of these timing criteria determine whether to wait for a measured value or proceed with imputation.

70. The computer-implemented method of claim 67, further comprising:
determining, by the processor, that a first input feature of the plurality of input features is in the second state;
determining, by the processor, whether a measurement order for the first input feature has been generated subsequent to determining that the first input feature is in the second state; and
generating, by the processor and in response to determining that the measurement order for the first input feature has not been generated, at least one of (1) a notification alerting that the measurement order for the first input feature is needed or (2) the measurement order for the first input feature.

71. The computer-implemented method of claim 67, wherein the clinical data is received as a continuous data stream during a monitoring period, and wherein the input features are continuously updated as new clinical data is received.

72. The computer-implemented method of claim 67, further comprising:
imputing, by the processor, a substitute value to a second input feature of the plurality of input features prior to generating the prediction using the machine learning model.

73. The computer-implemented method of claim 72, wherein the substitute value associated with the second input feature is determined, by the processor, using a second machine learning model, and wherein a second plurality of input features to the second machine learning model used to calculate the substitute value are the plurality of input features except for the second input feature.

74. The computer-implemented method of claim 67, wherein the determining, by the processor, whether each of the plurality of input features is in the first state or the second state is performed repeatedly at predetermined intervals until either all input features are in the first state or a maximum trigger time limit is exceeded from the time of the request.

75. The computer-implemented method of claim 67, wherein the plurality of input features includes at least one input feature of a first group, at least one input feature of a second group, and at least one input feature of a third group,
wherein the first group includes input features in the group consisting of: age, sex at birth, race, ethnicity, past medical history of the patient, current complaints or symptoms of the patient, neurological assessments, clinical decision support alerts, clinician or other chart notes, diagnosis codes, procedures performed on the patient, current medications of the patient, interventions, patient care setting, medical imaging data or assessments, electrograms, endoscopic tests, systolic blood pressure, diastolic blood pressure, body temperature, respiratory rate, heart rate, blood oxygen saturation, and a fraction of inspired oxygen,
wherein the second group includes input features in the group consisting of: white blood cell count, lymphocyte count, neutrophil count, platelet count, creatinine, blood urea nitrogen, potassium, chloride, total carbon dioxide, sodium, albumin, bilirubin, lactate, glucose, calcium, hematocrit, hemoglobin concentration, lab-derived biomarkers, clinical severity measures and composites, biopsies data, and microorganism test data, and
wherein the third group includes input features in the group consisting of: procalcitonin, C-reactive protein, protein biomarkers, genomic biomarkers, gene expression or transcriptomic biomarkers, and composite biomarker scores.

76. The computer-implemented method of claim 67, further comprising:
receiving, by the processor, a request to trigger the machine learning model to generate the prediction, wherein the processor begins reading patient data stored in a memory upon receiving the request, wherein the patient data includes the at least some of the plurality of input features, and wherein the at least some of the plurality of input features are received in real-time as the at least some of the plurality of input features are measured.

77. The computer-implemented method of claim 67, further comprising:
receiving, by the processor, an indication that a measured value of a first input feature of the plurality of input features is expected to be received at a first time outside of a predetermined time window; and
adjusting, by the processor, the predetermined time window to generate an adjusted time window, wherein the first time is within the adjusted time window.

78. A system, comprising:
a memory; and
a processor in communication with the memory, the processor configured to perform operations including:
receiving clinical data associated with a plurality of input features of a machine learning model configured to generate a prediction of the risk of the patient having or developing the medical condition;
determining whether each of the plurality of input features is in a first state or a second state, wherein the first state and the second state are indicative of whether an input feature of the plurality of input features is available for triggering the machine learning model, and wherein each of the plurality of input features has a respective set of requirements to be in the first state; and
if every input feature in the plurality of input features is in a first state, generating, using the machine learning model, the prediction of the risk of the patient having or developing the medical condition.

79. The system of claim 78, wherein determining whether each input feature is in the first state or second state comprises evaluating, for each input feature:
(a) whether the input feature has a resulted observation taken within an acceptable time interval,
(b) whether the input feature is imputable based on an imputable flag,
(c) whether an observation request exists within an observation request limit relative to a current time,
(d) whether a result time limit has been exceeded from a time observation was requested, and/or
(e) whether a maximum trigger time has been exceeded from receipt of the request, wherein the first state requires either a resulted observation within the acceptable time interval or satisfaction of imputation criteria based on the imputable flag and associated time limits.

80. The system of claim 78, wherein determining the first state or second state for an input feature comprises: for non-imputable input features, requiring a resulted observation within the acceptable time interval, for imputable input features, determining the state based on a combination of: (i) presence or absence of an observation request within the observation request limit, (ii) elapsed time relative to the result time limit when an observation request exists, and (iii) current time relative to the observation request limit when no observation request exists, wherein different combinations of these timing criteria determine whether to wait for a measured value or proceed with imputation.

81. The system of claim 78, wherein the operations further include:
determining that a first input feature of the plurality of input features is in the second state;
determining whether a measurement order for the first input feature has been generated subsequent to determining that the first input feature is in the second state; and
automatically generating the order for the first input feature and in response to determining that the measurement order for the first input feature has not been generated, at least one of (1) a notification alerting that the measurement order for the first input feature is needed or (2) the measurement order for the first input feature.

82. The system of claim 78, wherein the clinical data is received as a continuous data stream during a monitoring period, and wherein the input features are continuously updated as new clinical data is received.

83. A non-transitory, computer-readable medium storing instructions, which when executed by a processor, cause the processor to perform operations including:
receiving clinical data associated with a plurality of input features of a machine learning model configured to generate a prediction of the risk of the patient having or developing the medical condition;
determining whether each of the plurality of input features is in a first state or a second state, wherein the first state and the second state are indicative of whether an input feature of the plurality of input features is available for triggering the machine learning model, and wherein each of the plurality of input features has a respective set of requirements to be in the first state; and
if every input feature in the plurality of input features is in a first state, generating, using the machine learning model, the prediction of the risk of the patient having or developing the medical condition.

84. The non-transitory, computer-readable medium of claim 83, wherein determining whether each input feature is in the first state or second state comprises evaluating, for each input feature:
(a) whether the input feature has a resulted observation taken within an acceptable time interval,
(b) whether the input feature is imputable based on an imputable flag,
(c) whether an observation request exists within an observation request limit relative to a current time,
(d) whether a result time limit has been exceeded from a time observation was requested, and/or
(e) whether a maximum trigger time has been exceeded from receipt of the request, wherein the first state requires either a resulted observation within the acceptable time interval or satisfaction of imputation criteria based on the imputable flag and associated time limits.

85. The non-transitory, computer-readable medium of claim 83, wherein determining the first state or second state for an input feature comprises: for non-imputable input features, requiring a resulted observation within the acceptable time interval, for imputable input features, determining the state based on a combination of: (i) presence or absence of an observation request within the observation request limit, (ii) elapsed time relative to the result time limit when an observation request exists, and (iii) current time relative to the observation request limit when no observation request exists, wherein different combinations of these timing criteria determine whether to wait for a measured value or proceed with imputation.

86. The non-transitory, computer-readable medium of claim 83, wherein the operations further include:
determining that a first input feature of the plurality of input features is in the second state;
determining whether a measurement order for the first input feature has been generated subsequent to determining that the first input feature is in the second state; and
automatically generating the order for the first input feature and in response to determining that the measurement order for the first input feature has not been generated, at least one of (1) a notification alerting that the measurement order for the first input feature is needed or (2) the measurement order for the first input feature.
